SEQUENCE LISTING (28 sequences)

SEQ ID NO: 1 - protein, 92 aa, Homo Sapiens
GSEVSDKRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 2 - protein, 68 aa, Homo Sapiens
SPYSSDTTPCCFAYIARPLPRAHIKEYFYTSGKCSNPAVVFVTRKNRQVCANPEKKWVREYINSLEMS

SEQ ID NO: 3 - protein, 74 aa, Homo Sapiens
GPASVPTTCCFNLANRKIPLQRLESYRRITSGKCPQKAVIFKTKLAKDICADPKKKWVQDSMKYLDQKSPTPKP

SEQ ID NO: 4 - protein, 73 aa, Homo Sapiens
KSMQVPFSRCCFSFAEQEIPLRAILCYRNTSSICSNEGLIFKLKRGKEACALDTVGWVQRHRKMLRHCPSKRK

SEQ ID NO: 5 - protein, 76 aa, Homo Sapiens
QPDAINAPVTCCYNFTNRKISVQRLASYRRITSSKCPKEAVIFKTIVAKEICADPKQKWVQDSMDHLDKQTQTPKT

SEQ ID NO: 6 - protein, 76 aa, Homo Sapiens
QPVGINTSTTCCYRFINKKIPKQRLESYRRTTSSHCPREAVIFKTKLDKEICADPTQKWVQDFMKHLDKKTQTPKL

SEQ ID NO: 7 - protein, 82 aa, Homo Sapiens
GPDAVSTPVTCCYNVVKQKIHVRKLKSYRRITSSQCPREAVIFRTILDKEICADPKEKWVKNSINHLDKTSQTFILEPSCLG

SEQ ID NO: 8 - protein, 70 aa, Homo Sapiens
ASLAADTPTACCFSYTSRQIPQNFIADYFETSSQCSKPGVIFLTKRSRQVCADPSEEWVQKYVSDLELSA

SEQ ID NO: 9 - protein, 69 aa, Homo Sapiens
APMGSDPPTACCFSYTARKLPRNFVVDYYETSSLCSQPAVVFQTKRSKQVCADPSESWVQEYVYDLELN

SEQ ID NO: 10 - protein, 70 aa, Homo Sapiens
ASNFDCCLGYTDRILHPKFIVGFTRQLANEGCDINAIIFHTKKKLSVCANPKQTWVKYIVRLLSKKVKNM

SEQ ID NO: 11 - protein, 77 aa, Homo Sapiens
GTNDAEDCCLSVTQKPIPGYIVRNFHYLLIKDGCRVPAVVFTTLRGRQLCAPPDQPWVERIIQRLQRTSAKMKRRSS

SEQ ID NO: 12 - protein, 74 aa, Homo Sapiens
GDTLGASWHRPDKCCLGYQKRPLPQVLLSSWYPTSQLCSKPGVIFLTKRGRQVCADKSKDWVKKLMQQLPVTAR

SEQ ID NO: 13 - protein, 99 aa, Homo Sapiens
RVTKDAETEFMMSKLPLENPVLLDRFHATSADCCISYTPRSIPCSLLESYFETNSECSKPGVIFLTKKGRRFCANPSDKQVQVCMRMLKLDTRIKTRKN

SEQ ID NO: 14 - protein, 97 aa, Homo Sapiens
QPKVPEWVNTPSTCCLKYYEKVLPRRLVVGYRKALNCHLPAIIFVTKRNREVCTNPNDDWVQEYIKDPNLPLLPTRNLSTVKIITAKNGQPQLLNSQ

SEQ ID NO: 15 - protein, 74 aa, Homo Sapiens
TKTESSSRGPYHPSECCFTYTTYKIPRQRIMDYYETNSQCSKPGIVFITKRGHSVCTNPSDKWVQDYIKDMKEN

SEQ ID NO: 16 - protein, 111 aa, Homo Sapiens
SDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 17 - protein, 69 aa, Homo Sapiens
GPYGANMEDSVCCRDYVRYRLPLRVVKHFYWTSDSCPRPGVVLLTFRDKEICADPRVPWVKMILNKLSQ

SEQ ID NO: 18 - protein, 71 aa, Homo Sapiens
ARGTNVGRECCLEYFKGAIPLRKLKTWYQTSEDCSRDAIVFVTVQGRAICSDPNNKRVKNAVKYLQSLERS

SEQ ID NO: 19 - protein, 127 aa, Homo Sapiens
QGVFEDCCLAYHYPIGWAVLRRAWTYRIQEVSGSCNLPAAIFYLPKRHRKVCGNPKSREVQRAMKLLDARNKVFAKLHHNMQTFQAGPHAVKKLSSGNSKLSSSKFSNPISSSKRNVSLLISANSGL

SEQ ID NO: 20 - protein, 71 aa, Homo Sapiens
KPVSLSYRCPCRFFESHVARANVKHLKILNTPACALQIVARLKNNNRQVCIDPKLKWIQEYLEKALNRFKM

SEQ ID NO: 21 - protein, 77 aa, Homo Sapiens
VPLSRTVRCTCISISNQPVNPRSLEKLEIIPASQFCPRVEIIATMKKKGEKRCLNPESKAIKNLLKAVSKEMSKRSP

SEQ ID NO: 22 - protein, 77 aa, Homo Sapiens
AVLPRSAKELRCQCIKTYSKPFHPKFIKELRVIESGPHCANTEIIVKLSDGRELCLDPKENWVQRVVEKFLKRAENS

SEQ ID NO: 23 - protein, 103 aa, Homo Sapiens
TPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 24 - protein, 77 aa, Homo Sapiens
GPVSAVLTELRCTCLRVTLRVNPKTIGKLQVFPAGPQCSKVEVVASLKNGKQVCLDPEAPFLKKVIQKILDSGNKKN

SEQ ID NO: 25 - protein, 73 aa, Homo Sapiens
ASVATELRCQCLQTLQGIHPKNIQSVNVKSPGPHCAQTEVIATLKNGRKACLNPASPIVKKIIEKMLNSDKSN

SEQ ID NO: 26 - protein, 73 aa, Homo Sapiens
APLATELRCQCLQTLQGIHLKNIQSVKVKSPGPHCAQTEVIATLKNGQKACLNPASPMVKKIIEKMLKNGKSN

SEQ ID NO: 27 - protein, 73 aa, Homo Sapiens
ASVVTELRCQCLQTLQGIHLKNIQSVNVRSPGPHCAQTEVIATLKNGKKACLNPASPMVQKIIEKILNKGSTN

SEQ ID NO: 28 - protein, 76 aa, Homo Sapiens
QHHGVTKCNITCSKMTSKIPVALLIHYQQNQASCGKRAIILETRQHRLFCADPKEQWVKDAMQHLDRQAAALTRNG

(19) United States
(12) Patent Application Publication
(10) Pub. No.: US 2004/0077835 A1
(43) Pub. Date: Apr. 22, 2004

(54) Chemokine receptor modulators, production and use

(76) Inventors: Robin Offord, Bernex (CH); Hubert Gaertner, Archamps (FR); Oliver Hartley, Geneva (CH)

Correspondence Address: LINIAK, BERENATO & WHITE, LLC, 6550 ROCK SPRING DRIVE, SUITE 240, BETHESDA, MD, 20817, US

(21) Appl. No.: 10/332,038

(22) PCT Filed: Jul. 12, 2001
(86) PCT No.: PCT/US01/21934

(51) Int. Cl. A61K 38/19; C07K 14/52

(52) U.S. Cl. 530/351; 424/85.1

(57) ABSTRACT

Chemokine receptor modulators comprising a chemically modified carboxyl-terminus (C-terminus) and/or amino-terminus (N-terminus) for modulating potency and pharmacokinetic properties, and methods of production and use are disclosed. The compounds and methods of the invention are exemplified by novel N-terminal, C-terminal and N/C-terminal analogs of CC and CXC chemokines. The chemokine receptor modulator analogs of the invention are useful for the treatment of disorders involving the naturally occurring chemokine that the analogs of the invention antagonize, such as for the treatment of HIV and AIDS related disorders and for the treatment of asthma, allergic rhinitis, atopic dermatitis, atheroma/atherosclerosis, organ transplant rejection, and rheumatoid arthritis.

### CROSS-REFERENCE TO RELATED APPLICATIONS

This application is a continuation-in-part of U.S. patent application Ser. No. 60/217,683 (filed Jul. 12, 2000), herein incorporated by reference.

### TECHNICAL FIELD

The invention relates to chemokine receptor modulators, and methods for their production and use.

### BACKGROUND OF THE INVENTION

Chemokines are small proteins involved in leukocyte trafficking and various other biological processes. Most chemokines localize and enhance inflammation by inducing chemotaxis and cell activation of different types of inflammatory cells typically present at inflammatory sites. Some chemokines have properties apart from chemotaxis, such as inducing the proliferation and activation of killer cells, modulating growth of haematopoietic progenitor cell types, trafficking of haematopoietic progenitor cells in and out of the bone marrow in inflammatory conditions, angiogenesis and tumor growth. (See, e.g., Baggiolini et al., Ann. Rev. Immunology (1997) 15:675-705; Zlotnik et al., Critical Rev. Immunology (1999) 19(1):1-4; Wang et al., J. Immunological Methods (1998) 220(1-2):1-17; and Moser et al., Intl. Rev. Immunology (1998) 16(3-4):323-344).

The amino acid sequence, structure and function of many chemokines are known. Chemokines have molecular masses of about 8-10 kDa and show approximately 20-50 percent sequence homology among each other at the protein level. The proteins also share common tertiary structures. All chemokines possess a number of conserved cysteine residues involved in intramolecular disulfide bond formation, which are utilized to identify and classify chemokines. For instance, chemokines having the first two cysteine residues separated by a single amino acid are called “C-X-C” chemokines (also called “alpha” chemokines). Chemokines having the first two cysteine residues adjacent are called “CC” chemokines (also called “beta” chemokines). The “C” chemokines differ from the other chemokines by the absence of a cysteine residue (also called “gamma” chemokines). The C chemokines show similarity to some members of the CC chemokines but have lost the first and third cysteine residues that are characteristic of the CC and CXC chemokines. Members of the small group of chemokines with the first two cysteine residues separated by three amino acid are called “CXXXC” chemokines (also called “CX₃C” or “delta” chemokines). There are subgroups of chemokines as well. For instance, CC chemokines containing two additional conserved cysteine residues are known, and sometimes the term “C6-beta” chemokine is used for this subgroup. Most chemokines identified to date are members of the CC and CXC chemokine classes.

The biological activities of chemokines are mediated by receptors. This includes chemokine-specific receptors as well as receptors with overlapping ligand specificity that bind several different chemokines belonging to either the CC chemokines or the group of CXC chemokines. For instance, the CC chemokine SDF-1α is specific for the CXCR4 receptor, whereas the CXC chemokine RANTES binds to the CCR1, CCR3 and CCR5 receptors. Another example is the chemokine Eotaxin, which is a ligand for the CCR3 (also known as CKR3) receptors. (See, e.g., Cyster, J. G., Science (1999) 286:2098-2102; Ponath et al., J. Experimental Medicine (1996) 183(6):2437-2448; Ponath et al., J. Clinical Investigation (1996) 97(3):604-12; and Yamada et al., Biochem. Biophys. Res. Communications (1997) 231(2): 365-368.

Chemokines have been implicated in important disease pathways, such as asthma, allergic rhinitis, atopic dermatitis, cancer, viral diseases, atheroma/atheroschleosis, rheumatoid arthritis and organ transplant rejection. However, a general problem with many chemokines and their potential use as therapeutics relates to their inherent property of promoting or aggravating leukocyte inflammatory responses and infection. To this end, numerous modifications of chemokines have been made in an attempt to generate antagonists of the corresponding wild type chemokine. A classic and representative example is the situation for RANTES. Under certain conditions, wild type RANTES can enhance inflammation and HIV infection (Gordon et al., J. Virol. (1999) 73:684-694; Czaplewski et al., J. Biol. Chem. (1999) 274:16077-16084). In contrast, substitutions at positions 26 (E26A) and 66 (E66S) of the RANTES polypeptide chain convert the molecule to its non-inflammatory version and improve its ability to compete with its receptors for HIV (Appay et al., J. Biol. Chem. (1999) 274(39):27505-27512). Moreover, N-terminal modifications of RANTES have been made that result in antagonists that can block HIV- 1 infection, including N-terminal truncation [RANTES 9-68], addition of methionine (“Met-RANTES”), aminooxypentane (“AOP-RANTES”), or nonanoyl (“NNY-RANTES”) (Arenzana-Seisdedos, et al., Nature (1996) 383:400; Mack, et al., J. Exp. Med. (1998) 187:1215-1224; Proudfoot, et al., J. Biol. Chem. (1996) 271:2599-2603; Wells, et al., WO 96/17935; Simmons, et al., Science (1997) 276:276-279; Offord et al., WO 99/11666; and Mosier et al., J. Virology (1999) 73(5):3544-3550).

While such approaches have improved antagonist-associated potency in some cases, one of the challenges in making chemokine receptor modulators is increasing potency while improving other drug properties such as pharmacokinetics. Also, finding a general strategy and method for making potent antagonists of chemokines and the corresponding chemokine receptor modulator compounds and their use in the preparation of medicaments for use in prevention and/or treatment of disease is desired. The present invention addresses these and other needs.

### SUMMARY OF THE INVENTION

The invention is directed to amino-terminal (“N-terminal”) and carboxyl-terminal (“C-terminal”) modified chemokine receptor modulators that inhibit the action of the corresponding naturally occurring chemokine. The N-terminal chemokine receptor modulators of the invention comprise a chemokine polypeptide chain modified at its N-terminus with a aliphatic chain and one or more amino acid derivatives. The C-terminal chemokine receptor modulators of the invention comprise a chemokine polypeptide chain modified at its C-terminus with a aliphatic chain or polycyclic. The N- and C-terminal chemokine receptor modulators of the invention also may include modifications at both the N- and C-termini in combination. Also provided are methods of production and use of the chemokine receptor modulators. The present invention is significant in that it provides a general approach for making compounds that are potent inhibitors of the corresponding naturally occurring wild type chemokines or their receptors.

In detail, the invention concerns a chemokine receptor modulator comprising a chemokine polypeptide chain modified at its N-terminus with an aliphatic chain and one or more amino acid derivatives.

The invention particularly concerns the embodiment of such chemokine receptor modulators wherein the chemokine polypeptide chain comprises an amino acid sequence that is substantially homologous to the amino acid sequence of a naturally occurring wild type chemokine (such as a CC chemokine, a CXC chemokine, etc).

The invention further concerns the embodiment of such chemokine receptor modulators wherein the N-terminus comprises amino acids of the chemokine polypeptide chain that are N-terminal to the first disulfide-forming cysteine of the chemokine polypeptide chain.

The invention further concerns the embodiment of such chemokine receptor modulators wherein the aliphatic chain is a hydrocarbon chain comprising 5 to 26 carbons, and/or wherein the amino acid derivative has the formula —(N-CnR-CO)—, where n is 1-22, R is hydrogen, alkyl or aromatic, and where N and Cn, N and R, or Cn and R can form a cyclic structure.

The invention further concerns a chemokine receptor modulator comprising a chemokine polypeptide chain modified at its C-terminus with an aliphatic chain (especially wherein the aliphatic chain comprises 5 to 22 carbons)or polycyclic, especially wherein the aliphatic chain or polycyclic is a lipid.

The invention further concerns a chemokine receptor modulator comprising a chemokine polypeptide chain modified at its N-terminus with an aliphatic chain and one or more amino acid derivatives, and at its C-terminus with an aliphatic chain or polycyclic.

The invention further concerns a pharmaceutical composition comprising a chemokine receptor modulator, wherein the chemokine receptor modulator comprises a chemokine polypeptide chain modified at its N-terminus with an aliphatic chain and one or more amino acid derivatives, or a pharmaceutically acceptable salt thereof.

The invention further concerns a pharmaceutical composition comprising a chemokine receptor modulator, wherein the chemokine receptor modulator comprises a chemokine polypeptide chain modified at its C-terminus with an aliphatic chain or polycyclic, or a pharmaceutically acceptable salt thereof.

The invention further concerns a pharmaceutical composition comprising a chemokine receptor modulator comprising a chemokine polypeptide chain modified at its N-terminus with an aliphatic chain and one or more amino acid derivatives, and at its C-terminus with an aliphatic chain or polycyclic, or a pharmaceutically acceptable salt thereof.

The invention further concerns a pharmaceutical composition comprising a method of treating a disease state (especially wherein the disease state is an inflammatory disease, or wherein the disease state is caused or associated with HIV infection) in a mammal (including humans) that is alleviated by treatment with a chemokine receptor modulator, which method comprises administering to a mammal in need of such a treatment a therapeutically effective amount of a chemokine receptor modulator, wherein the chemokine receptor modulator comprises a chemokine polypeptide chain (A) modified at its N-terminus with an aliphatic chain and one or more amino acid derivatives, (B) modified at its C-terminus with an aliphatic chain or polycyclic, or (C) modified at its N-terminus with an aliphatic chain and one or more amino acid derivatives, and at its C-terminus with an aliphatic chain or polycyclic.

### BRIEF DESCRIPTION OF THE DRAWINGS

FIG. 1 is a schematic showing a general structure of four classes of naturally occurring chemokines and their corresponding N-terminal, N-loop and C-terminal regions as defined by conserved cysteine patterns, where “C” is one letter code for cysteine and “X” represents any amino acid other than cysteine.

FIGS. 2A-2E depict examples of naturally occurring amino acid sequences of various chemokine polypeptide chains, including the corresponding N-terminal, N-loop and C-terminal regions of these chemokines. The standard one letter amino acid code for the 20 genetically encoded amino acids is used.

### DESCRIPTION OF THE PREFERRED EMBODIMENTS

The invention is directed to N- and C-terminal chemokine receptor modulators. As used herein, the term “chemokine receptor modulator” is intended to refer to a polypeptide, or derivatized polypeptide that modulate or inhibit the activity of a naturally occurring chemokine as determined by a suitable chemokine bioassay. Such inhibitors may act by antagonizing one or more properties of a chemokine receptor to which they bind (e.g., inhibiting viral infection, causing receptor down-modulation, causing receptor internalization) and thereby “antagonize” the normal cycle of receptor recyling back to the cell surface. In the context of other biological responses, such modulators can act as agonists of a receptor, e.g., inducing calcium flux, initiating chemotaxis, etc. Thus, the chemokine receptor modulators of the present invention can act as antagonists (including partial antagonism), but also may act as agonists (including partial agonists), or mixtures of both. Preferred are chemokine receptor modulators that exhibit at least one antagonistic property, i.e., an ability to antagonize one or more biological properties of a chemokine receptor to which they bind (e.g., block or partially block (1) viral infection, (2) chemotaxis, (3) receptor cycling etc.). Such chemokine receptor modulators may act by binding to (or engaging), but not activating, a chemokine's receptor, or may mediate their action by other means.

The N-terminal chemokine receptor modulators of the present invention comprise a chemokine polypeptide chain modified at its N-terminus with an aliphatic chain and one or more amino acid derivatives. The N-terminal chemokine receptor modulators have, as read in the N-terminal to C-terminal direction, the following formula: J1-X1-Z1-CHEMOKINE, where: J1 is an aliphatic chain; X1 is a spacer comprising zero or more amino acids of the N-terminal amino acid sequence of the chemokine polypeptide chain; Z1 is an amino acid derivative; CHEMOKINE is the remaining amino acid sequence of the chemokine polypeptide chain; and the dashes (“-”) represent a covalent bond. The compounds are designed to respect the overall length of the N-terminal region of the polypeptide chain. Accordingly, depending upon the length of the aliphatic chain and the position of the amino acid derivative, the N-terminal antagonist may include one or more substitutions, insertions or deletions at the N-terminus relative to the corresponding naturally occurring chemokine polypeptide chain.

The C-terminal chemokine receptor modulators comprise a chemokine polypeptide chain modified at its C-terminus with an aliphatic chain or polycyclic. These compounds have, as read in the N-terminal to C-terminal direction, the following formula: CHEMOKINE-X2-J2, where: X2 is a spacer comprising zero or more amino acids of the C-terminal amino acid sequence of the chemokine polypeptide chain; J2 is an aliphatic chain or polycyclic; CHEMOKINE is the remaining amino acid sequence of the chemokine polypeptide chain; and the dashes (“-”) represent a covalent bond. The C-terminal region of chemokines is amenable to substantive modification, including insertion, deletion or addition of one or more amino acids or other chemical moieties to extend the C-terminal end of the polypeptide chain compared to the corresponding wild type molecule, as well as addition of fluorescent labels and biocompatible polymers, and conjugation to other compounds such as small organic molecules, peptides, proteins and the like.

The N- and C-terminal chemokine receptor modulator of the invention may include modifications at both the N- and C-terminal regions, which when referred to specifically are designated as N-/C-terminal chemokine receptor modulators. These compounds have the formula J1-X1-Z1-CHEMOKINE-X2-J2, where: J1, X1, Z1, CHEMOKINE, X2, J2 and “-” are as described above. These compounds combine the advantages of the N- and C-terminal modifications in a synergistic manner depending on a given end use

By “chemokine polypeptide chain” is intended a polypeptide chain that is substantially homologous to the polypeptide chain of a naturally occurring wild type chemokine. By “N-terminal amino acid sequence” is intended the amino acid sequence of the chemokine polypeptide chain that is adjacent and N-terminal to the first disulfide-forming cysteine of the naturally occurring chemokine polypeptide chain. By “C-terminal amino acid sequence” is intended the amino acid sequence of the chemokine polypeptide chain that is adjacent and C-terminal to the last disulfide-forming cysteine of the naturally occurring chemokine polypeptide chain. The chemokine polypeptide chain, the N-terminal amino acid sequence, the C-terminal amino acid sequence, and the first and last disulfide-forming cysteines forming the basis of a chemokine receptor modulator of the invention can be readily deduced from the corresponding amino acid sequence of the naturally occurring chemokine, as well as by homology modeling with other chemokines of the same class, such as comparison to the amino acid sequences of the known C, CC, CXC and CXXXC chemokines.

For instance, the following are examples of known naturally occurring chemokines, many of which have been described under different names and thus appear several times: 6Ckine, 9E3, ATAC, ABCD-1, ACT-2, ALP, AMAC-1, AMCF-1, AMCF-2, AIF, ANAP, Angie, beta-R1, Beta-Thromboglobulin, BCA-1, BLC, blr-1 ligand, BRAK, C10, CCF18, Ck-beta-6, Ck-beta-8, Ck-beta-8-1, Ck-beta-10, Ck-beta-11, cCAF, CEF-4, CINC, C7, CKA-3, CRG-2, CRG-10, CTAP-3, DC-CK1, ELC, Eotaxin, Eotaxin-2, Exodus-1, Exodus-2, ECIP-1, ENA-78, EDNAP, ENAP, FIC, FDNCF, FINAP, Fractalkine, G26, GDCF, GOS-19-1, GOS-19-2, GOS-19-3, GCF, GCP-2, GCP-2-like, GRO1, GRO2, GRO3, GRO-alpha, GRO-beta, GRO-gamma, H400, HC-11, HC-14, HC-21, HCC-1, HCC-2, HCC-3, HCC4 H174, Heparin neutralizing protein, Humig, 1-309, ILINCK, I-TAC, Ifi10, IL8, IP-9, IP-10, IRH, JE, KC, Lymphotactin, L2G25B, LAG-1, LARC, LCC-1, LD78-alpha, LD78-beta, LD78-gamma, LDCF, LEC, Lkn-1, LMC, LAI, LCF, LA-PF4, LDGF, LDNAP, LIF, LIX, LUCT, Lungkine, LYNAP, Manchester inhibitor, MARC, MCAF, MCP-1, MCP-2, MCP-3, MCP-4, MCP-5, MDC, MIP-1-alpha, MIP-1-beta, MIP-1-delta, MIP-1-gamma, MIP-3, MIP-3-alpha, MIP-3-beta, MIP-4, MIP-5, Monotactin-1, MPIF-1, MPIF-2, MRP-1, MRP-2, M119, MDNAP, MDNCF, Megakaryocyte-stimulatory-factor, MGSA, Mig, MIP-2, mob-1, MOC, MONAP, NC28, NCC-1, NCC-2, NCC-3, NCC-4 N51, NAF, NAP-1, NAP-2, NAP-3, NAP-4, NAP S, NCF, NCP, Neurotactin, Oncostatin A, P16, P500, PARC, pAT464, pAT744, PBP, PBP-like, PBSF, PF4, PF4-like, PF4-ALT, PF4V1, PLF, PPBP, RANTES, SCM-1-alpha, SCI, SCY A26, SLC, SMC-CF, ST38, STCP-1, SDF-1-alpha, SDF-1-beta, TARC, TCA-3, TCA-4, TDCF, TECK, TSG-8, TY5, TCF, TLSF-alpha, TLSF-beta, TPAR-1, TSG-1.

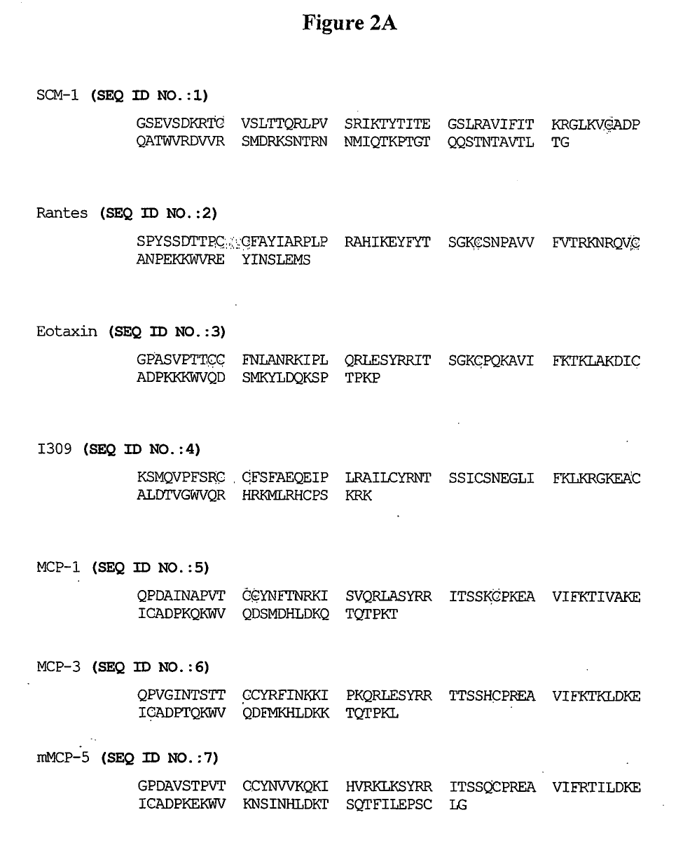

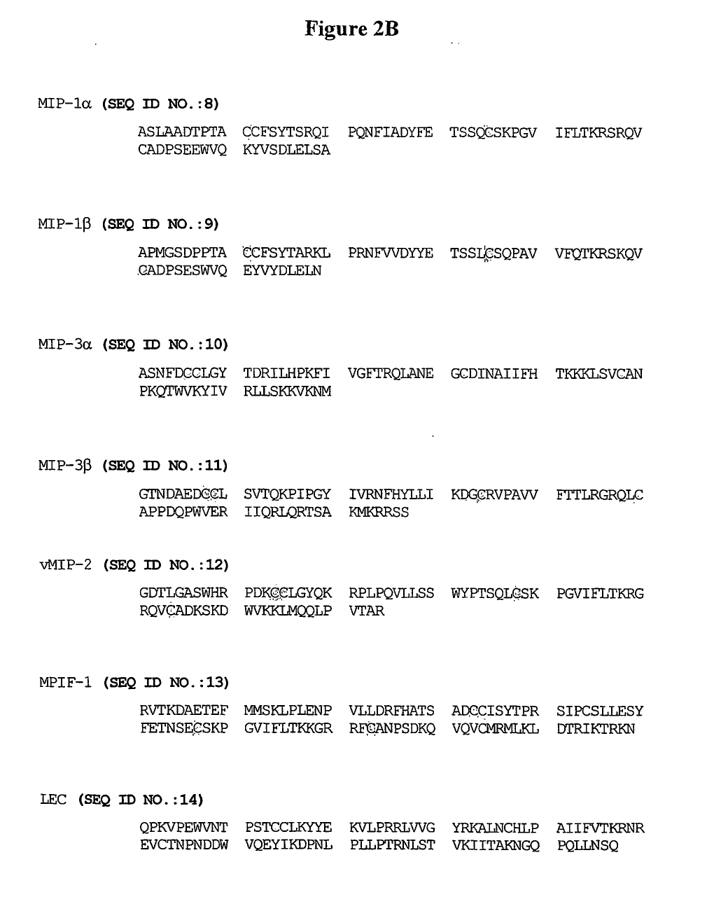

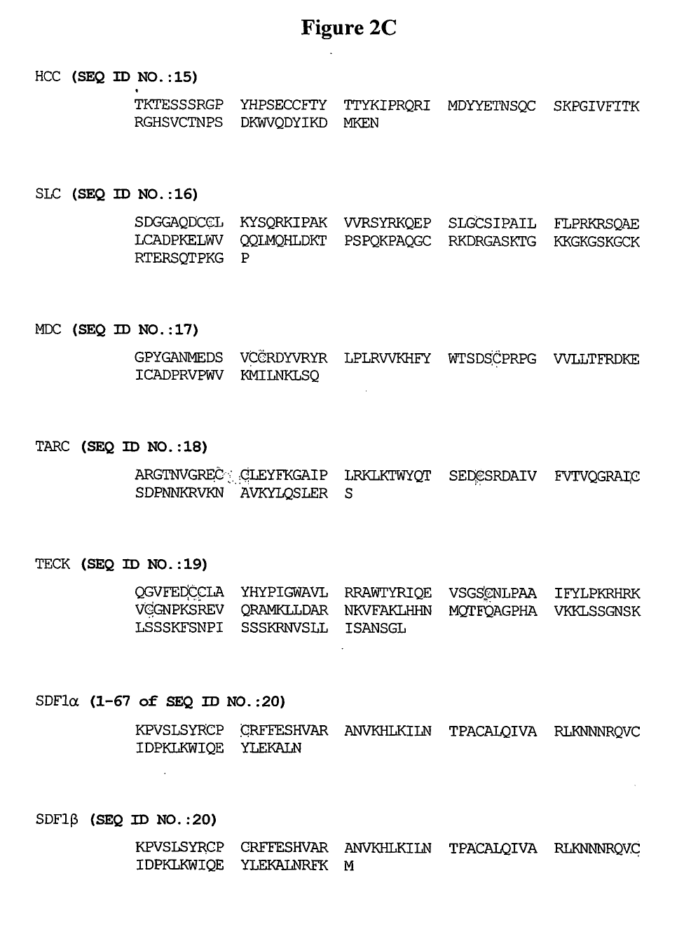

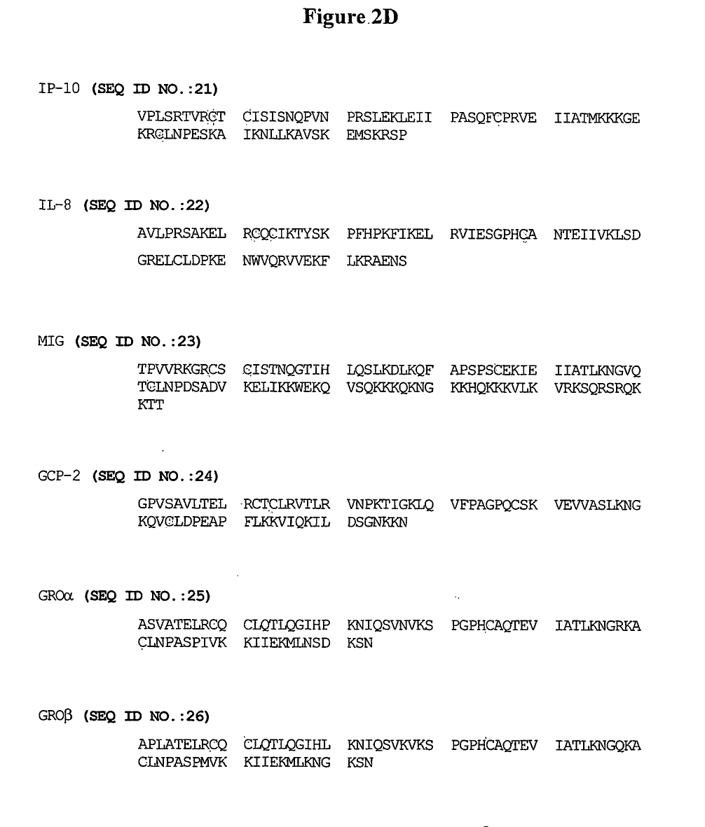

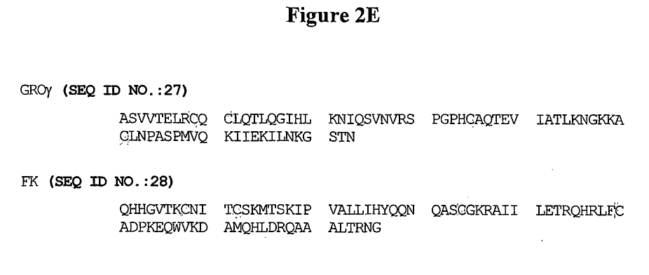

By way of illustration, and not by way of limitation, examples of some of the above-listed wild type chemokine polypeptide chains and their corresponding N-terminal, N-loop and C-terminal amino acid sequences are depicted in FIGS. 2A-2E. As can be appreciated, additional chemokine polypeptide chains are known and obtainable from many different sources including publicly accessible databases such as the Genome Database (Johns Hopkins University, Maryland USA), Protein Data Bank (Brookhaven National Laboratory & Rutgers University, New Jersey USA), Entrez (National Institutes of Health, Maryland USA), NRL 3D (Pittsburgh Supercomputing Center, Carnegie Mellon University, Pennsylvania USA), CATH (University College London, London, UK), NIH Gopher Server (NIH, Maryland USA), ProLink (Boston University, Massachusetts USA), The Nucleic Acid Database (Rutgers University, New Jersey USA), Genebank (National Library of Medicine, Maryland USA), Expasy (Swiss Institute of Bioinformatics, Geneva Switzerland), and the like. Also, new chemokines, such as those derived from various gene and protein sequencing programs can be identified by homology and pattern matching following standard techniques known in the art, including databases and associated tools for achieving this purpose.

In one embodiment of the present invention, directed evolution techniques, such as phage display or modular shuffling, may be used to generate chemokines with increased receptor specificity. The testing of chemokine derivatives or analogues for their ability to bind chemokine receptors using phage display has been described in the treatment and prevention of HIV (U.S. Pat. No. 6,214,540; DeVico et al.). Phage display techniques have also been used to detect or identify ligands, inhibitors or promoters of receptor proteins for CXC Chemokine Receptor 3 (CXCR3) (U.S. Pat. No. 6,140,064, Loetscher et al.), which are characterized by selective binding of one or more chemokines with the ability to induce a cellular response (U.S. Pat. No. 6,184,358, Loetscher et al.). The use of phage display has been described in the labeling and selection of molecules (U.S. Pat. No. 6,180,336, Osbourn et al.), the labeling and subsequent purification of binding molecules for specific antigens (see e.g., WO92/01047), and in the determination of peptide composition for prevention and treatment of HIV infection and immune disorders (U.S. Pat. No. 6,090,388, Wang).

Phage display procedures involving G protein-coupled receptors have also been described (see e.g., Doorbar, J. et al., “Isolation of a peptide antagonist to the thrombin receptor using phage display,” J. Mol. Biol., 244: 361-9 (1994)), with preferred regions for directed evolution at the N-loop region (Konigs, C, “2 Monoclonal antibody screening of a phage-displayed random peptide library reveals mimotopes of chemokine receptor CCR5: implications for the tertiary structure of the receptor and for an N-terminal binding site for HIV-1 gp120,” Eur. J. Immunol. 2000 Apr.; 30(4): 1162-71; Sidhu, S. S. et al., “High copy display of large proteins on phage for functional selections,” J Mol Biol 2000 Feb. 18;296(2):487-95; Fielding, A. K. et al., “A hyperfusogenic gibbon ape leukemia envelope glycoprotein: targeting of a cytotoxic gene by ligand display,” Hum Gene Ther 2000 Apr. 10;11(6):817-26), the region between N-loop and C-terminus, and the C-terminus (Cain, S. A. et al. “Selection of novel ligands from a whole-molecule randomly mutated C5a library,” Protein Eng 2001 Mar.;14(3):189-93; Heller, T. et al., “Selection of a C5a receptor antagonist from phage libraries attenuating the inflammatory response in immune complex disease and ischemia/reperfusion injury,” J. Immunol. 1999 Jul. 15;163(2):985-94; Chang, C. et al., “Dissection of the LXXLL nuclear receptor-coactivator interaction motif using combinatorial peptide libraries: discovery of peptide antagonists of estrogen receptors alpha and beta,” Mol Cell Biol 1999 Dec.;19(12):8226-39).

Suitable aliphatic chains of J1 and J2 include, but are not limited to, aliphatic chains that are five (C5) to twenty-two (C22) carbons in length. The chain may be unsaturated and/or unbranched, or may have variable degrees of saturation and/or branching. The aliphatic chains have the general formula Cn(Rm)-, where Cn is the number of carbons and Rm is the number of substituent groups selected from hydrogen, alkyl, acyl, aromatic or combination(s) thereof, and n and m may be the same or different. The J1 and J2 groups are joined to X1, X2 or to the chemokine polypeptide chain via any suitable covalent linkage. Examples of suitable covalent linkages include, but are not limited to: amide, ketone, aldehyde, ester, ether, thioether, thioester, thiozolidine, oxime, oxizolidine, Schiff-base and Schiff-base type linkages (for example, hydrazide). Without limitation, such linkages can comprise:

—C(O)—NH—(CH₂)—C(O)—; —C(O)—NH—(CH₂)ₓ—C(O)—; —C(O)—NH—(CH₂)—NH—C(O)—; —C(O)—NH—(CH₂)ₓ—NH—C(O)—; —C(O)—NH—(CH₂)—[(CH₂)—NH]_{y}—C(O)—; —C(O)—NH—(CH₂)—[(CH₂)ₓ—NH]_{y}—C(O)—; —C(O)—NH—(CH₂)—NH—CH₂—C(O)—; —C(O)—NH—(CH₂)—NH—(CH₂)ₓ—C(O)—; —C(O)—NH—(CH₂)—[NH—(CH₂)ₓ]_{y}—C(O)—; —C(O)—NH—(CH₂)—[NH—(CH₂)]_{y}—C(O)—;

—NH—(CH₂)—C(O)—; —NH—(CH₂)ₓ—C(O)—; —NH—(CH₂)—NH—C(O)—; —NH—(CH₂)ₓ—NH—C(O)—; —NH—(CH₂)—[(CH₂)—NH]_{y}—C(O)—; —NH—(CH₂)—[(CH₂)—NH]_{y}—C(O)—; —NH—(CH₂)—NH—CH₂—C(O)—; —NH—(CH₂)—NH—(CH₂)ₓ—C(O)—; —NH—(CH₂)—[NH—(CH₂)ₓ]_{y}—C(O)—; —NH—(CH₂)—[NH—(CH₂)]_{y}—C(O)—;

—ONH—C(O)—; —ONH—(CH₂)—C(O)—; —ONH—(CH₂)ₓ—C(O)—; —ONH—(CH₂)—NH—C(O)—; —ONH—(CH₂)—(CH₂)—NH—C(O)—; —ONH—(CH₂)ₓ—NH—C(O)—; —ONH—(CH₂)—[(CH₂)—NH]_{y}—C(O)—; —ONH—(CH₂)—[(CH₂)ₓ—NH]_{y}—C(O)—; —ONH—(CH₂)—NH—CH₂—C(O)—; —ONH—(CH₂)—NH—(CH₂)ₓ—C(O)—; —ONH—(CH₂)—[NH—(CH₂)ₓ]_{y}—C(O)—; —ONH—(CH₂)—[NH—(CH₂)]_{y}—C(O)—;

—OCH₂—C(O)—; —OCH₂—(CH₂)—C(O)—; —OCH₂—(CH₂)ₓ—C(O)—; —OCH₂—(CH₂)—NH—C(O)—; —OCH₂—(CH₂)—(CH₂)—NH—C(O)—; —OCH₂—(CH₂)ₓ—NH—C(O)—; —OCH₂—(CH₂)—[(CH₂)—NH]_{y}—C(O)—; —OCH₂—(CH₂)—[(CH₂)ₓ—NH]_{y}—C(O)—; —OCH₂—(CH₂)—NH—CH₂—C(O)—; —OCH₂—(CH₂)—NH—(CH₂)ₓ—C(O)—; —OCH₂—(CH₂)—[NH—(CH₂)]_{y}—C(O)—; —OCH₂—(CH₂)—[NH—(CH₂)]_{y}—C(O)—; —OCH₂—NH—C(O)—; —OCH₂—NH—(CH₂)—C(O)—; —OCH₂—NH—(CH₂)ₓ—C(O)—; —OCH₂—NH—(CH₂)—NH—C(O)—; —OCH₂—NH—(CH₂)—(CH₂)—NH—C(O)—; —OCH₂—NH—(CH₂)ₓ—NH—C(O)—; —OCH₂—NH—(CH₂)—[(CH₂)—NH]_{y}—C(O)—; —OCH₂—NH—[(CH₂)ₓ—NH]_{y}—C(O)—; —OCH₂—(CH₂)—NH—CH₂—C(O)—; —OCH₂—(CH₂)—NH—(CH₂)ₓ—C(O)—; —OCH₂—(CH₂)—[NH—(CH₂)ₓ]_{y}—C(O)—; —OCH₂—(CH₂)—[NH—(CH₂)]_{y}—C(O)—; —OCH₂—N(CH₃)—C(O)—; —OCH₂—N(CH₃)—(CH₂)—C(O)—; —OCH₂—N(CH₃)—(CH₂)ₓ—C(O)—; —OCH₂—N(CH₃)—(CH₂)—NH—C(O)—; —OCH₂—N(CH₃)—(CH₂)ₓ—NH—C(O)—; —OCH₂—N(CH₃)—(CH₂)ₓ—NH—C(O)—; —OCH₂—N(CH₃)—(CH₂)—[(CH₂)—NH]_{y}—C(O)—; —OCH₂—N(CH₃)—(CH₂)—[(CH₂)ₓ—NH]_{y}—C(O)—; —OCH₂—N(CH₃)—(CH₂)—NH—CH₂—C(O)—; —OCH₂—N(CH₃)—(CH₂)—NH—(CH₂)ₓ—C(O)—; —OCH₂—N(CH₃)—(CH₂)—[NH—(CH₂)ₓ]_{y}—C(O)—; —OCH₂—N(CH₃)—(CH₂)—[NH—(CH₂)]_{y}—C(O)—;

—O—C(O)—C(O)—; —O—C(O)—(CH₂)—C(O)—; —O—C(O)—(CH₂)ₓ—C(O)—; —O—C(O)—(CH₂)—NH—C(O)—; —O—C(O)—(CH₂)—(CH₂)—NH—C(O)—; —O—C(O)—(CH₂)ₓ—NH—C(O)—; —O—C(O)—(CH₂)—[(CH₂)—NH]_{y}—C(O)—; —O—C(O)—(CH₂)—[(CH₂)ₓ—NH]_{y}—C(O)—; —O—C(O)—(CH₂)—NH—CHₓ—C(O)—; —O—C(O)—(CH₂)—NH—(CH₂)ₓ—C(O)—; —O—C(O)—(CH₂)—[NH—(CH₂)ₓ]_{y}—C(O)—; —O—C(O)—(CH₂)—[NH—(CH₂)]_{y}—C(O)—; —O—C(O)—NH—C(O)—; —O—C(O)—NH—(CH₂)—C(O)—; —O—C(O)—NH—(CH₂)ₓ—C(O)—; —O—C(O)—NH—(CH₂)—NH—C(O)—; —O—C(O)—NH—(CH₂)—(CH₂)—NH—C(O)—; —O—C(O)—NH—(CH₂)ₓ—NH—C(O)—; —O—C(O)—NH—(CH₂)—[(CH₂)—NH]_{y}—C(O)—; —O—C(O)—NH—[(CH₂)ₓ—NH]_{y}—C(O)—; —O—C(O)—(CH₂)—NH—CH₂—C(O)—; —O—C(O)—(CH₂)—NH—(CH₂)ₓ—C(O)—; —O—C(O)—(CH₂)—[NH—(CH₂)ₓ]_{y}—C(O)—; —O—C(O)—(CH₂)—[NH—(CH₂)]_{y}—C(O)—; —O—C(O)—N(CH₃)—C(O)—; —O—C(O)—, N(CH₃)—(CH₂)—C(O)—; —O—C(O)—N(CH₃)—(CH₂)ₓ—C(O)—; —O—C(O)—N(CH₃)—(CH₂)—NH—C(O)—; —O—C(O)—N(CH₃)—(CH₂)ₓ—NH—C(O)—; —O—C(O)—N(CH₃)—(CH₂)ₓ—NH—C(O)—; —O—C(O)—N(CH₃)—(CH₂)—[(CH₂)—NH]_{y}—C(O)—; —O—C(O)—N(CH₃)—(CH₂)—[(CH₂)_{y}—NH]_{y}—C(O)—; —O—C(O)—N(CH₃)—(CH₂)—NH—CH₂—C(O)—; —O—C(O)—N(CH₃)—(CH₂)—NH—(CH₂)ₓ—C(O)—; —O—C(O)—N(CH₃)—(CH₂)—[NH—(CH₂)ₓ]y—C(O)—; —O—C(O)—N(CH₃)—(CH₂)—[NH—(CH₂)]_{y}—C(O)—;

—CH═CH—C(O)—; —CH═CH—(CH₂)—C(O)—; —CH═CH—(CH₂)ₓ—C(O)—; —CH═CH—(CH₂)—NH—C(O)—; —CH═CH—(CH₂)ₓ—NH—C(O)—; —CH═CH—(CH₂)—[(CH₂)—NH]_{y}—C(O)—; —CH═CH—(CH₂)—[(CH₂)ₓ—NH]_{y}—C(O)—; —CH═CH—(CH₂)—NH—CH₂—C(O)—; —CH═CH—(CH₂)—NH—(CH₂)ₓ—C(O)—; —CH═CH—(CH₂)—[NH—(CH₂)]_{y}—C(O)—; —CH═CH—(CH₂)—[NH—(CH₂)ₓ]_{y}—C(O)—;

—SCH₂—N(CH₃)—C(O)—; —SCH₂—N(CH₃)—(CH₂)—C(O)—; —SCH₂—N(CH₃)—(CH₂)ₓ—C(O)—; —SCH₂—N(CH₃)—(CH₂)—NH—C(O)—; —SCH₂—N(CH₃)—(CH₂)ₓ—NH—C(O)—; —SCH₂—N(CH₃)—(CH₂)ₓ—NH—C(O)—; —SCH₂—N(CH₃)—(CH₂)—[(CH₂)—NH]_{y}—C(O)—; —SCH₂—N(CH₃)—(CH₂)—[(CH₂)ₓ—NH]_{y}—C(O)—; —SCH₂—N(CH₃)—(CH₂)—NH—CH₂—C(O)—; —SCH₂—N(CH₃)—(CH₂)—NH—(CH₂)ₓ—C(O)—; —SCH₂—N(CH₃)—(CH₂)—[NH—(CH₂)ₓ]_{y}—C(O)—; —SCH₂—N(CH₃)—(CH₂)—[NH—(CH₂)]_{y}—C(O)—;

—S—C(O)—C(O)—; —S—C(O)—(CH₂)—C(O)—; —S—C(O)—(CH₂)ₓ—C(O)—; —S—C(O)—(CH₂)—NH—C(O)—; —S—C(O)—(CH₂)—(CH₂)—NH—C(O)—; —S—C(O)—(CH₂)ₓ—NH—C(O)—; —S—C(O)—(CH₂)—[(CH₂)—NH]_{y}—C(O)—; —S—C(O)—(CH₂)—[(CH₂)ₓ—NH]_{y}—C(O)—; —S—C(O)—(CH₂)—NH—CH₂—C(O)—; —S—C(O)—(CH₂)—NH—(CH₂)ₓ—C(O)—; —S—C(O)—(CH₂)—[NH—(CH₂)ₓ]_{y}—C(O)—; —S—C(O)—(CH₂)—[NH—(CH₂)]_{y}—C(O)—; —S—C(O)—NH—C(O)—; —S—C(O)—NH—(CH₂)—C(O)—; —S—C(O)—NH—(CH₂)ₓ—C(O)—; —S—C(O)—NH—(CH₂)—NH—C(O)—; —S—C(O)—NH—(CH₂)—(CH₂)—NH—C(O)—; —S—C(O)—NH—(CH₂)ₓ—NH—C(O)—; —S—C(O)—NH—(CH₂)—[(CH₂)—NH]_{y}—C(O)—; —S—C(O)—NH—[(CH₂)ₓ—NH]_{y}—C(O)—; —S—C(O)—(CH₂)—NH—CH₂—C(O)—; —S—C(O)—(CH₂)—NH—(CH₂)ₓ—C(O)—; —S—C(O)—(CH₂)—[NH—(CH₂)ₓ]_{y}—C(O)—; —S—C(O)—(CH₂)—[NH—(CH₂)]_{y}—C(O)—; —S—C(O)—N(CH₃)—C(O)—; —S—C(O)—N(CH₃)—(CH₂)—C(O)—; —S—C(O)—N(CH₃)—(CH₂)ₓ—C(O)—; —S—C(O)—N(CH₃)—(CH₂)—NH—C(O)—; —S—C(O)—N(CH₃)—(CH₂)ₓ—NH—C(O)—; —S—C(O)—N(CH₃)—(CH₂)ₓ—NH—C(O)—; —S—C(O)—N(CH₃)—(CH₂)—[(CH₂)—NH]_{y}—C(O)—; —S—C(O)—N(CH₃)—(CH₂)—[(CH₂)ₓ—NH]_{y}—C(O)—; —S—C(O)—N(CH₃)—(CH₂)—NH—CH₂—C(O)—; —S—C(O)—N(CH₃)—(CH₂)—NH—(CH₂)ₓ—C(O)—; —S—C(O)—N(CH₃)—(CH₂)—[NH—(CH₂)ₓ]_{y}—C(O)—; —S—C(O)—N(CH₃)—(CH₂)—[NH—(CH₂)]_{y}—C(O)—; —C₃H₆SN—C(O)—; —C₃H₆SN—(CH₂)—C(O)—; —C₃H₆SN—(CH₂)ₓ—C(O)—; —C₃H₆SN—(CH₂)—NH—C(O)—; —C₃H₆SN—(CH₂)—(CH₂)—NH—C(O)—; —C₃H₆SN—(CH₂)ₓ—NH—C(O)—; —C₃H₆SN—(CH₂)—[(CH₂)—NH]_{y}—C(O)—; —C₃H₆SN—(CH₂)—[(CH₂)ₓ—NH]_{y}—C(O)—; —C₃H₆SN—(CH₂)—NH—CH₂—C(O)—; —C₃H₆SN—(CH₂)—NH—(CH₂)ₓ—C(O)—; —C₃H₆SN—(CH₂)—[—(CH₂)ₓ]_{y}—C(O)—; —C₃H₆SN—(CH₂)—[NH—(CH₂)]_{y}—C(O)—; —C₃H₆SN—NH—C(O)—; —C₃H₆SN—NH—(CH₂)—C(O)—; —C₃H₆SN—NH—(CH₂)ₓ—C(O)—; —C₃H₆SN—NH—(CH₂)—NH—C(O)—; —C₃H₆SN—NH—(CH₂)—(CH₂)—NH—C(O)—; —C₃H₆SN—NH—(CH₂)ₓ—NH—C(O)—; —C₃H₆SN—NH—(CH₂)—[(CH₂)—NH]_{y}—C(O)—; —C₃H₆SN—NH—[(CH₂)ₓ—NH]_{y}—C(O)—; —C₃H₆SN—(CH₂)—NH—CH₂—C(O)—; —S—C(O)—(CH₂)—NH—(CH₂)ₓ—C(O)—; —C₃H₆SN—(CH₂)—[NH—(CH₂)ₓ]_{y}—C(O)—; —C₃H₆SN—(CH₂)—[NH—(CH₂)]_{y}—C(O)—; —C₃H₆SN—N(CH₃)—C(O)—; —C₃H₆SN—N(CH₃)—(CH₂)—C(O)—; —C₃H₆SN—N(CH₃)—(CH₂)ₓ—C(O)—; —C₃H₆SN—N(CH₃)—(CH₂)—NH—C(O)—; —C₃H₆SN—N(CH₃)—(CH₂)ₓ—NH—C(O)—; —C₃H₆SN—N(CH₃)—(CH₂)ₓ—NH—C(O)—; —C₃H₆SN—N(CH₃)—(CH₂)—[(CH₂)—NH]_{y}—C(O)—; —C₃H₆SN—N(CH₃)—(CH₂)—[(CH₂)ₓ—NH]_{y}—C(O)—; —C₃H₆SN—N(CH₃)—(CH₂)—NH—CH₂—C(O)—; —C₃H₆SN—N(CH₃)—(CH₂)—NH—(CH₂)ₓ—C(O)—; —C₃H₆SN—N(CH₃)—(CH₂)—[NH—(CH₂)ₓ]_{y}—C(O)—; —C₃H₆SN—N(CH₃)—(CH₂)—[NH—(C2)]_{y}—C(O)—;

—C₃H₆ON—C(O)—; —C₃H₆ON—(CH₂)—C(O)—; —C₃H₆ON—(CH₂)ₓ—C(O)—; —C₃H₆ON—(CH₂)—NH—C(O)—; —C₃H₆ON—(CH₂)—(CH₂)—NH—C(O)—; —C₃H₆ON—(CH₂)ₓ—NH—C(O)—; —C₃H₆ON—(CH₂)—[(CH₂)—NH]_{y}—C(O)—; —C₃H₆ON—(CH₂)—[(CH₂)ₓ—NH]_{y}—C(O)—; —C₃H₆ON—(CH₂)—NH—CH₂—C(O)—; —C₃H₆ON—(CH₂)—NH—(CH₂)ₓ—C(O)—; —C₃H₆ON—(CH₂)—[NH—(CH₂)ₓ]Y—C(O)—; —C₃H₆ON—(CH₂)—[NH—(CH₂)]_{y}—C(O)—; —C₃H₆ON—NH—C(O)—; —C₃H₆ON—NH—(CH₂)—C(O)—; —C₃H₆ON—NH—(CH₂)ₓ—C(O)—; —C₃H₆ON—NH—(CH₂)—NH—C(O)—; —C₃H₆ON—NH—(CH₂)—(CH₂)—NH—C(O)—; —C₃H₆ON—NH—(CH₂)ₓ—NH—C(O)—; —C₃H₆ON—NH—(CH₂)—[(CH₂)—NH]_{y}—C(O)—; —C₃H₆ON—NH—[(CH₂)ₓ—NH]_{y}—C(O)—; —C₃H₆ON—(CH₂)—NH—CH₂—C(O)—; —S—C(O)—(CH₂)—NH—(CH₂)ₓ—C(O)—; —C₃H₆ON—(CH₂)—[NH—(CH₂)ₓ]_{y}—C(O)—; —C₃H₆ON—(CH₂)—[NH—(CH₂)]_{y}—C(O)—; —C₃H₆ON—N(CH₃)—C(O)—; —C₃H₆ON—N(CH₃)—(CH₂)—C(O)—; —C₃H₆ON—N(CH₃)—(CH₂)ₓ—C(O)—; —C₃H₆ON—N(CH₃)—(CH₂)—NH—C(O)—; —C₃H₆ON—N(CH₃)—(CH₂)ₓ—NH—C(O)—; —C₃H₆ON—N(CH₃)—(CH₂)ₓ—NH—C(O)—; —C₃H₆ON—N(CH₃)—(CH₂)—[(CH₂)—NH]_{y}—C(O)—; —C₃H₆ON—N(CH₃)—(CH₂)—[(CH₂)ₓ—NH]_{y}—C(O)—; —C₃H₆ON—N(CH₃)—(CH₂)—NH—CH₂—C(O)—; —C₃H₆ON—N(CH₃)—(CH₂)—NH—(CH₂)ₓ—C(O)—; —C₃H₆ON—N(CH₃)—(CH₂)—[NH—(CH₂)ₓ]_{y}—C(O)—; —C₃H₆ON—N(CH₃)—(CH₂)—[NH—(CH₂)]_{y}—C(O)—;

—O—C(O)—; —C(O)—, or a covalent bond, where x and y are 2, 3, 4 or more, and may be the same or different.

Chemistries suitable for linkage systems are well known and can be utilized for this purpose (see, for example, “Chemistry of Protein Conjugation and Cross-Linking”, S. S. Wong, Ed., CRC Press, Inc. (1993); Perspectives in Bioconjugate Chemistry, Claude F. Modres, Ed., ACS (1993)).

In addition to joining J1 and J2 to X1, X2 or the chemokine polypeptide chain, the linkage system employed can be selected to tune the physical-chemical and/or biological properties of the target molecule, provided that the resulting molecule retains its antagonist properties. This can be accomplished, for example, by incorporating a linkage system that is more (or less) stable under one type of condition compared to another for modulating half-life and the like, or for tuning potency, specificity and the like by utilizing linkage systems of variable length, rigidity, charge and/or chirality. The linkage unit joining the hydrocarbon chains to the chemokine polypeptide chain can vary substantially, with the proviso that the overall length and space filling of J1 and/or J2 will most preferably approximate that of the naturally occurring chemokine.

In a preferred embodiment, the aliphatic chain J1 is a hydrocarbon chain five (C₅) to ten (C10) carbons in length, and the aliphatic chain J2 is a lipid 12 (C₁₂) to twenty (C₂₀) carbons in length. Examples of the J1 C5-C10 hydrocarbon chains include, but are not limited to: —C₅H₁₁, —C₅H₉, —C₅H₇, —C₅H₅, —C₅H₃, —C₆H₁₃, —C₆H₁₁, —C₆H₉, —C₆H₇, —C₆H₅, —C₆H₃, —C₇H₁₅, —C₇H₁₃, —C₇H₁₁, —C₇H₉, —C₇H₇, —C₇H₅, —C₇H₃, —C₈H₁₇, —C₈H₁₅, —C₈H₁₃, —C₈H₁₁, —C₈H₉, —C₈H₇, —C₈H₅, —C₈H₃, —C₉H₁₉, _{C9}H₁₇, —C₉H₁₅, —C₉H₁₃, —C₉H₁₁, —C₉H₉, —C₉H₇, —C₉H₅, —C₉H₃, —C₁₀H₂₁, —C₁₀H₁₉, C₁₀H₁₇, —C₁₀H₁₅, —C₁₀H₁₃, —C₁₀H₁₁, —C₁₀H₉, —C₁₀H₇, —C₁₀H₅, and —C₁₀H₃.

Suitable J2 lipids include, but are not limited to the fatty acid derived lipids and polycyclic steroid derived lipids. The fatty acids include, but are not limited to, saturated and unsaturated fatty acids. Examples of saturated fatty acids are lauric acid (C12), myristic acid (C14), palmitic acid (C16), steric acid (C18), and arachidic acid (C20). Examples of unsaturated fatty acids include oleic acid (C18), linoleic acid (C18), linolenic acid (C18), eleosteric acid (C18), and arachidonic acid (C20). The polycyclics include, but are not limited to: aldosterone, cholestanol, cholesterol, cholic acid, coprostanol, corticosterone, cortisone, dehydrocholesterol, desmosterol, digitogenin, ergosterol, estradiol, hydoxycorticosterone, lathosterol, prednisone, pregnenolone, progesterone, testosterone, zymosterol, etc. The fatty acids are usually joined to the chemokine polypeptide chain through the acid component, thereby yielding an acyl-linked moiety, although other linkages may be employed. The linkage unit joining the hydrocarbon chains to the chemokine polypeptide chain can vary substantially, with the proviso that the overall length and space filling of the N-terminal region approximates that of the naturally occurring chemokine. The C-terminal region has been found to be more flexible in this regard, so the overall length and space filling can be varied to a greater extent than with the N-terminal region.

In another preferred embodiment, the J1 and J2 components when comprised in a chemokine derivative of the invention comprise a C5 to C20 saturated or unsaturated acyl chain, such as nonanoyl, nonenoyl, aminooxypentane, dodecanoyl, myristoyl, palmitate, lauryl, palmitoyl, eicosanoyl, oleoyl, or cholyl. For example, the J1 substituent can be nonaoyl or aminooxypentane and the J2 substituent can be a saturated or unsaturated fatty acid, preferably a C12-C20 fatty acid, or a polycyclic steroid lipid such as cholesterol.

Depending upon the nature and length of the aliphatic chain, the chemokine receptor modulators of the invention may include additional amino acids or other moieties that are added to the polypeptide chain, particularly at the C-terminal end to provide a spacer group and/or separate attachment site for the aliphatic moiety.

By “amino acid derivative” is intended an amino acid or amino acid-like chemical entity other than one of the 20 genetically encoded naturally occurring amino acids. In particular, the amino acid derivative Z1 is other than one of the 20 30 genetically encoded naturally occurring amino acids, and has the formula —(N-CnR-CO)—, where Cn is 1-22 carbons, R is hydrogen, alkyl or aromatic, and where N and Cn, N and R, or Cn and R can form a cyclic structure. Also, N, Cn and R can each have one or more hydrogens in its reduced form depending on the amino acid derivative. The alkyl moiety can be substituted or non-substituted, its can be linear, branched, or cyclic, and may include one or more heteroatoms. The aromatic can be substituted or non-substituted, and include one or more heteroatoms. The amino acid derivatives can be made de novo or obtained from commercial sources (See, e.g., Calbiochem-Novabiochem AG, Switzerland; Advanced Chemtech, Louisville, Ky., USA; Lancaster Synthesis, Inc., Windham, N.H., USA; Bachem California, Inc., Torrance, Calif., USA; Genzyme Corp., Cambridge, Mass., USA). Examples of amino acid derivatives include, but are not liited to, aminoisobutyric acid (Aib), hydroxyproline (Hyp), 1,2,3,4-tetrahydroisoquinoline-3-COOH (Tic), indoline-2-carboxylic acid (indol), 4-difluoro-proline (P(4,4DiF)), L-thiazolidine-4-carboxylic acid (Thz), L-homoproline (HoP), 3,4-dehydro-proline (ΔPro), 3,4dihydroxyphenylalanine (F(3,4-DiOH)), pBzl,-3,4dihydroxyphenylalanine (F(3,4-DiOH, pBzl)), benzophenone (p-Bz), cyclohexyl-alanine (Cha), 3-(2-naphtyl)-alanine (βNal), cyclohexyl-glycine (Chg), and phenylglycine (Phg).

With respect to X1, CHEMOKINE and X2, the amino acid sequence of these components is substantially homologous to the corresponding naturally occurring wild type molecule. The term “substantially homologous” when used herein includes amino acid sequences having at least 40%, 50%, 60%, 70%, 80%, 90%, 95% or 99% sequence homology with the given sequence (95-99% preference). This term can include, but is not limited to, amino acid sequences having from 1 to 20, from 1 to 10 or from 1 to 5 single amino acid deletions, insertions or substitutions relative to a given sequence provided that the resultant polypeptide acts as an antagonist of the corresponding naturally occurring chemokine.

For instance, it is well known in the art that certain amino acids can be replaced with others resulting in no substantial change in the properties of a polypeptide, including but not limited to conservative substitutions of amino acids. Such possibilities are within the scope of the present invention. It should also be noted that deletions or insertions of amino acids can often be made which do not substantially change the properties of a polypeptide. The present invention includes such deletions or insertions (which may be, for example up to 10, 20 or 50% of the length of the specific antagonist's sequence of the corresponding naturally occurring chemokine). Moreover, chemokines may be subjected to substantial modifications, including mixing and matching different chemokine polypeptide segments to create additional diversity, such as the modular ‘cross-over’ synthesis approach described in WO 99/11655, which reference is incorporated herein in its entirety by reference.

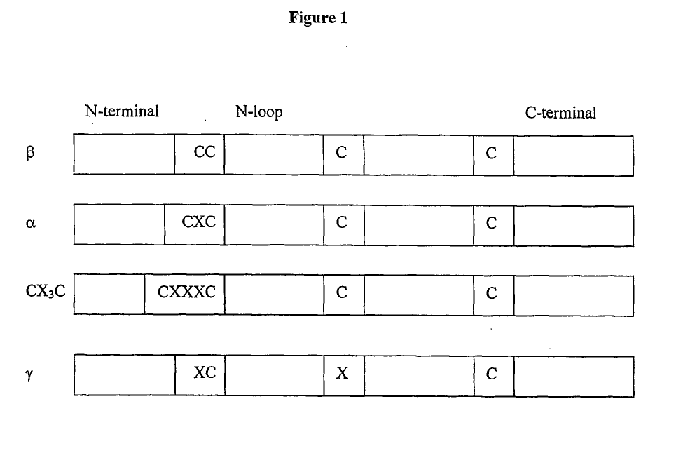

In addition to changes at the N- and C-termini, the chemokine receptor modulators of the invention also may include one or more amino acid substitutions, insertions or deletions elsewhere in the polypeptide chain, i.e., in the polypeptide chain represented in the above formulae by CHEMOKINE. In a preferred embodiment, changes are made in the N-loop of the chemokine to increase its specificity/selectivity for a target receptor. In this way, the N-loop modified chemokine receptor modulator blocks a specific receptor while minimizing the antagonist effect on other of its possible co-receptors. By “N-loop” is intended the 20 to 26 amino acid sequence region adjacent/C-terminal to the first conserved cysteine pattern defining the N-terminal region of a given chemokine polypeptide chain (see, FIGS. 1 and 2). For example, as read in the N- to C-terminal direction of the chemokine polypeptide chain, the N-loop of a CC chemokine is the region of amino acids located between and adjacent/C-terminal to the first and second conserved cysteine amino acids and adjacent/N-terminal to the third conserved cysteine amino acid.

The chemokine receptor modulators of the invention also may include a detectable label, such as a fluorophore, and other substituents introduced at specific, chosen sites, that convert the molecules into probes of the membrane and cell-biological events associated with chemokine action, virus inhibition and the like, as well as for monitoring pharmacokinetics and the like. The detectable labels are preferably attached to the C-terminal region of the chemokine receptor modulators. A detectable label may be incorporated during synthesis or post-synthesis of the chemokine polypeptide chain. As an example, a detectable label can be incorporated in a pre-ligation peptide segment during chain assembly, e.g., it may be convenient to conjugate a fluorophore to an unprotected reactive group on a resin-bound peptide before removal of other protecting groups and release of the labeled peptide from the resin. Amino acid derivatives comprising a detectable label and chemical synthesis techniques used to incorporate them into a peptide or polypeptide sequence are well known, and can be used for this purpose. In this way the resulting chemokine polypeptide chain ligation product can be designed to contain one or more detectable labels at pre-specified positions of choice. Alternatively, a detectable label can be added to reactive groups, preferably chemoselective reactive groups such as keto or aldehyde groups that permit site-specific attachment, present on a given amino acid of a peptide segment pre-ligation or even the polypeptide chain following ligation.

Detectable labels suitable for this purpose include photoactive groups, as well as chromophores including fluorophores and other dyes, or a hapten such as biotin. Such labels are available from many different commercial sources (See, e.g., Molecular Probes, Oregon USA; Sigma and affiliates, St. Louis, Mo., USA; and the like). For on resin labeling, Fluorescein, eosin, Oregon Green, Rhodamine Green, Rhodol Green, tetramethylrhodamine, Rhodamine Red, Texas Red, coumarin and NBD fluorophores, the dabcyl chromophore and biotin are all reasonably stable to hydrogen fluoride (HF), as well as to most other acids, and thus suitable for incorporation via solid phase synthesis. (Peled, et al., Biochemistry (1994) 33:7211; Ben-Efraim, et al., Biochemistry (1994) 33:6966). Other than the coumarins, these fluorophores also are stable to reagents used for de-protection of peptides synthesized using Fmoc chemistry (Strahilevitz, et al., Biochemistry (1994) 33:10951). The t-Boc and α-Fmoc derivatives of ε-dabcyl-L-lysine also can be used to incorporate the dabcyl chromophore at selected sites in a polypeptide sequence. The dabcyl chromophore has broad visible absorption and can used as a quenching group. The dabcyl group also can be incorporated at the N-terminus by using dabcyl succinimidyl ester (Maggiora, et al., J Med Chem (1992) 35:3727). EDANS is a common fluorophore for pairing with the dabcyl quencher in FRET experiments. This fluorophore is conveniently introduced during automated synthesis of peptides by using 5-((2-(t-Boc)-γ-glutamylaminoethyl) amino) naphthalene-1-sulfonic acid (Maggiora, et al., J. Med. Chem. (1992) 35:3727). An α-(t-Boc)-ε-dansyl-L-lysine can be used for incorporation of the dansyl fluorophore into polypeptides during chemical synthesis (Gauthier, et al., Arch Biochem. Biophys. (1993) 306:304). As with EDANS fluorescence of this fluorophore overlaps the absorption of dabcyl. Site-specific biotinylation of peptides can be achieved using the t-Boc-protected derivative of biocytin (Geahlen, et al., Anal. Biochem. (1992) 202:68), or other well known biotinylation derivatives such as NHS-biotin and the like. Racemic benzophenone phenylalanine analog also can be incorporated into peptides following its t-Boc or Fmoc protection (Jiang, et al., Intl. J. Peptide Prot. Res. (1995) 45:106). Resolution of the diastereomers can be accomplished during HPLC purification of the products; the unprotected benzophenone also can be resolved by standard techniques in the art. Keto-bearing amino acids for oxime coupling, aza/hydroxy tryptophan, biotyl-lysine and D-amino acids are among other examples of amino acids that can be utilized for on resin labeling. It will be recognized that other protected amino acids for automated peptide synthesis can be prepared by custom synthesis following standard techniques in the art. In another embodiment, the chemokine receptor modulators of the invention may include a drug conjugated thereto (See, e.g., WO 00/04926).

Also provided are methods of producing the chemokine receptor modulators of the invention. The method involves (i) synthesizing an analog of a naturally occurring chemokine that comprises a polypeptide chain having an amino acid sequence that is substantially homologous to the naturally occurring chemokine, where the polypeptide chain is modified at one or more of its N-terminus, N-loop and C-terminus with a moiety selected from an aliphatic chain and an amino acid derivative; and (ii) screening the chemokine analog for antagonist activity compared to the corresponding naturally occurring chemokine.

In particular, the method for production of the N-terminal chemokine receptor modulator comprises: (i) synthesizing an analog of a naturally occurring chemokine that comprises a polypeptide chain having an amino acid sequence that is substantially homologous to the naturally occurring chemokine, where the polypeptide chain is modified at its N-terminus with an aliphatic chain and one or more amino acid derivatives; and (ii) screening the chemokine analog for antagonist activity compared to the corresponding naturally occurring chemokine. The method for production of the C-terminal chemokine receptor modulator comprises: (i) synthesizing an analog of a naturally occurring chemokine that comprises a polypeptide chain having an amino acid sequence that is substantially homologous to the naturally occurring chemokine, where the polypeptide chain is modified at its C-terminus with an aliphatic chain or polycyclic; and (ii) screening the chemokine analog for antagonist activity compared to the naturally occurring chemokine. The method for production of the N-/C-terminal chemokine receptor modulators comprises: (i) synthesizing an analog of a naturally occurring chemokine that comprises a polypeptide chain having an amino acid sequence that is substantially homologous to the naturally occurring chemokine, where the polypeptide chain is modified at its N-terminus with an aliphatic chain and one or more amino acid derivatives, and is modified at its C-terminus with an aliphatic chain or polycyclic; and (ii) screening the chemokine analog for antagonist activity compared to the naturally occurring chemokine.

Synthesis of the chemokine receptor modulators of the invention is accomplished by chemical synthesis (i.e., ribosomal-free synthesis), or a combination of biological (i.e., ribosomal synthesis) and chemical synthesis. For chemical synthesis, the chemokine receptor modulators can be made in toto by stepwise chain assembly or fragment condensation techniques, such as solid or solution phase peptide synthesis using Fmoc and tBoc approaches, or by chemical ligation of peptide segments made in toto by chain assembly, or a combination of chain assembly and biological production. Such stepwise chain assembly or fragment condensation and ligation techniques are well known in the art (See, e.g., Kent, S. B. H., Ann. Rev. Biochem. (1988) 57:957-989; Dawson et al., Methods Enzymol. (1997) 287:3445; Muir et al., Methods Enzymol. (1997) 289:266-298; Wilken et al., Current Opinion in Biotechnology (1998) 9:412426; Ingenito et al., J. Amer. Chem. Soc. (1999) 121(49): 11369-11374; and Muir et al., Chemistry & Biology (1999) 6:R247-R256).

For chemical ligation, a first peptide segment having an N-terminal functional group is ligated to a second peptide segment having a C-terminal functional group that reacts with the N-terminal functional group to form a covalent bond therein between. Depending on the functional groups selected, the ligation reaction generates a product having a native amide bond or a non-native covalent bond at the ligation site. The first or second peptide segment employed for chemical ligation is typically made using stepwise chain assembly or fragment condensation. In particular, when the chemokine receptor modulators are made by ligation of peptide segments, the segments are made to contain the appropriate pendant chemoselective reactive groups with respect to the intended chemoselective reaction chemistry to be used for ligation. These chemistries include, but are not limited to, native chemical ligation (Dawson, et al., Science (1994) 266:776-779; Kent, et al., WO 96/34878), extended general chemical ligation (Kent, et al., WO 98/28434), oxime-forming chemical ligation (Rose, et al., J. Amer. Chem. Soc. (1994) 116:30-33), thioester forming ligation (Schnolzer, et al., Science (1992) 256:221-225), thioether forming ligation (Englebretsen, et al., Tet. Letts. (1995) 36(48):8871-8874), hydrazone forming ligation (Gaertner, et al., Bioconj. Chem. (1994) 5(4):333-338), and thiazolidine forming ligation and oxazolidine forming ligation (Zhang, et al., Proc. Natl. Acad. Sci. (1998) 95(16):9184-9189; Tam, et al., WO 95/00846).

Reaction conditions for a given ligation chemistry are selected to maintain the desired interaction of the ligation components. For example, pH and temperature, water-solubility of the peptides and components, ratio of peptides, water content and composition of the individual peptides can be varied to optimize ligation. Addition or exclusion of reagents that solubilize the peptides to different extents may further be used to control the specificity and rate of the desired ligation reaction. Reaction conditions are readily determined by assaying for the desired chemoselective reaction product compared to one or more internal and/or external controls.

A preferred method of chemical synthesis employs native chemical ligation, which is disclosed in Kent et al., WO 96/34878, and a method of preparing proteins chemically modified at the N- and/or C-terminal is disclosed in Offord et al., WO 99/11666, the disclosures of which are incorporated herein by reference. In general, a first peptide containing a C-terminal thioester is reacted with a second peptide with an N-terminal cysteine having an unoxidized sulfhydryl side chain. The unoxidized sulfhydryl side chain of the N-terminal cysteine is condensed with the C-terminal thioester in the presence of a catalytic amount of a thiol, preferably benzyl mercaptan, thiophenol, 2-nitrothiophenol, 2-thiobenzoic acid, 2-thiopyridine, and the like. An intermediate peptide is produced by linking the first and second peptides via a β-aminothioester bond, which rearranges to produce a peptide product comprising the first and second peptides linked by an amide bond.

For a combination of chemical and biological production, one peptide segment is made by chemical synthesis while the other is made using recombinant approaches, which segments are then joined using chemical ligation to generate the full-length product. For instance, intein expression systems can be utilized to exploit the inducible self-cleavage activity of an ‘intein’ protein-splicing element to generate a C-terminal thioester peptide segment. In particular, the intein undergoes specific self-cleavage in the presence of thiols such as DTT, b-mercaptoethanol or cysteine, which generates a peptide segment bearing a C-terminal thioester. (See, e.g., Muir et al., Chemistry & Biology (1999) 6:R247-R256; Chong et al., Gene (1997) 192:277-281; Chong et al., Nucl. Acids Res. (1998) 26:5109-5115; Evans et al., Protein Science (1998) 7:2256-2264; and Cotton et al., Chemistry & Biology (1999) 6(9):247-256). This C-terminal thioester bearing peptide segment may then be utilized to ligation a second peptide bearing an N-terminal thioester-reactive functionality, such as a peptide segment having an N-terminal cysteine as employed for native chemical ligation.

The aliphatic chains and amino acid derivatives can be incorporated during chain assembly, post chain assembly or a combination thereof. For incorporation during chain assembly, the amino acid derivatives and/or amino acids having an aliphatic chain attached thereto are incorporated in the stepwise or fragment condensation, and/or the ligation chain assembly process. These amino acids can be added in a stepwise fashion to the growing peptide chain during peptide synthesis, to assembled peptide segments targeted for ligation, or in some instances the pendant N- or C-terminal modifications can be provided by cleavage from a polymer support, whereby the cleavage product yields the desired aliphatic chain. For post chain assembly, amino acids or derivatives thereof having a reactive functional group are incorporated during chain assembly (in protected or unprotected form) which are then utilized in their unprotected reactive form for attachment of the desired moiety, i.e., in a post-peptide synthesis conjugation reaction. The post chain assembly attachment can be performed on a denatured linear peptide chain, or following folding of the polypeptide chain. In a preferred embodiment, the amino acid derivative is added during peptide synthesis at an amino acid position of interest, whereas the N-, C- and/or N-/C-terminal aliphatic chain is added following peptide synthesis through a conjugation reaction. Any of numerous conjugation chemistries can be utilized (See, e.g., Plaue, S et al., Biologicals. (1990) 18(3): 147-57; Wade, J. D. et al., Australas Biotechnol. (1993) 3(6):332-6; Doscher, M. S., Methods Enzymol. (1977) 47:578-617; Hancock, D. C. et al., Mol Biotechnol. (1995) 4(1):73-86; Albericio, F. et al., Methods Enzymol. (1997) 289: 313-36), as well as ligation chemistries, depending on the desired covalent linkage. Folding of the chemokine receptor modulators of the invention can be achieved following standard techniques in the art. See, e.g., WO 99/11655; WO 99/11666; Dawson et al., Methods Enzymol. (1997) 287:34-45).

For screening the synthesized chemokine compounds for antagonist activity, the compounds are examined by in vitro or in vivo based assays characterized by direct or indirect binding of the chemokine ligand to its corresponding receptor. Examples of chemokine receptors and their corresponding wild type chemokine include CXXXCR1 (Fractalkine); XCR1 (SCM-1); CXCR2 (GRO, LIX, MEP-2); CXCR3 (MIG, IP-10); CXCR4 (SDF-1); CXCR5 (BLC); CCR1 (MIP-1α, RANTES, MCP-3); CCR2 (MCP-1, MCP-3, MCP-5); CCR3 (Eotaxin, RNATES, MIP-1α); CCR4 (MDC, TARC); CCR5 (RANTES, MIP-1α, MIP-1β; CCR6 (MIP-3α); CCR7 (SLC, MIP-3β); CCR8 (TCA-3); and CCR9 (TECK). In vitro and in vivo assays for these systems are well know, and readily available or can be created de novo. See, e.g., U.S. Pat. Nos. 5,652,133; 5,834,419; WO 97/44054; WO 00/04926; and WO 00/0492. For instance, natural, transformed, and/or transgenic cell lines expressing one or more chemokine receptors are typically used to monitor the effect of chemokine-induced chemotaxis or the inhibition of this event when exposed to a chemokine receptor modulator, such as the compounds of the present invention. Animal models also may be employed, for example, to monitor a response profile in conjunction with treatment with a chemokine receptor modulator of the invention, or to characterize the pharmacokinetic and pharmacodynamic properties of the compounds. To characterize the compounds of the invention as inhibitors of viral infection, envelope-mediated cell fusion assays employing a target cell line and an envelop cell line may be employed for screening chemokine receptor modulators of the invention for their ability to prevent HIV infection. Of course cell-free viral infection assays may be employed as well for this purpose.

As an example, for assessing antagonism of chemotaxis in general, peripheral blood leukocytes can be employed, such as those isolated from normal donors according to established protocols for purification of monocytes, T lymphocytes and neutrophils. A panel of C, CC, CXXXC and CXC chemokine receptor-expressing test cells can be constructed and evaluated following exposure to serial dilutions of individual compounds of the invention. Native chemokines can be used as controls. For instance, a panel of cells transfected with expression cassettes encoding various chemokine receptors are suitable for this purposes. For instance, antagonist of chemokines such as RANTES, SDF-1α or SDF-1β and MIP can be screened using tranformants expression CXCR4/Fusion/LESTP, CCR3, CCR5, CXC₄ (such cells are available from various commercial and/or academic sources or can be prepared following standard protocols; see, e.g., Risau, et al., Nature 387:671-674 (1997); Angiololo, et al., Annals NY Acad. Sci. (1996) 795:158-167; Friedlander, et al., Science (1995) 870:1500-1502). The results can be expressed as the chemotaxis index (“CI”) representing the fold increase in the cell migration induced by stimuli versus control medium, and statistical significance determined.

Receptor binding assays also can be performed, for example, to evaluate competitive inhibition versus receptor recycling effects (see, Signoret, N. et al., “Endocytosis and recycling of the HIV coreceptor CCR5,” J Cell Biol. 2000 151(6):1281-94; Signoret, N. et al., “Analysis of chemokine receptor endocytosis and recycling,” Methods Mol Biol. 2000;138:197-207; Pelchen-Matthews, A. et al., “Chemokine receptor trafficking and viral replication,” Immunol Rev. 1999 April;168:33-49; Daugherty, B. L. et al., “Radiolabeled chemokine binding assays,” Methods Mol Biol. 2000;138:129-34; Mack, M. et al. “Downmodulation and recycling of chemokine receptors,” Methods Mol Biol. 2000;138:191-5; all herein incorporated by reference). This approach is well known and typically will employ labeled chemokine receptor modulators in the presence of increasing concentrations of unlabeled native chemokines following standard protocols. Of course labeling can be on either or both ligands. In this type of assay, the binding data can be analyzed, for example, with a computer program such as LIGAND (P. Munson, Division of Computer Research and Technology, NIH, Bethesda, Md.), and subjected to Scatchard plots analysis with both “one site” and “two site” models compared to native leukocytes or the panel of receptor-transfected cells expressing a target chemokine receptor. The rate of competition for binding by unlabeled ligands can then be calculated with the following formula: % inhibition=1−(Binding in the presence of unlabeled chemokine/binding in the presence of medium alone)×100.

For screening the compounds for their ability to prevent or alleviate viral infection and disease, the compounds can be screened against a panel of cells stably expressing either the appropriate receptor exposed to various viral strains and controls. For instance, U87/CD4 cells expressing CCR3, CCR5, CXC4 or CXCR4 receptors can be employed for screening infection of M-tropic, T-tropic and dual tropic HIV strains. Inhibition of viral infection can be be accessed as a percentage of infection relative to chemokine receptor modulator and control concentrations. See, e.g., McKnight, et al., Virology (1994) 201:8-18); and Mosier, et al., Science (1993) 260:689-692; Simmons, et al, Science (1997) 276:276-279; Wu, et al., J. Exp. Med. (1997) 185:168-169; and Trkola, et al., Nature (1996) 384:184-186). Calcium mobilization assays are another example useful for screening for antagonists of receptor binding, for instance to identify antagonists of native chemokines that are chemotactic for neutrophils and eosinophils (Jose, et al., J. Exp. Med. 179:881-887 (1994)). As another example, angiogenic activities of compounds of the invention can be evaluated by the chick chorioallantoic membrane (CAM) assay (Oikawa, et al., Cancer Lett. (1991) 59:57-66.

The chemokine receptor modulators of the invention have many uses, including use as research tools, diagnostics and as therapeutics. In particular, the chemokine receptor modulators of the invention have been found to possess valuable pharmacological properties, and have been shown to effectively block the inflammatory effects associated with the corresponding wild type molecules—which are involved in various disorders including asthma, allergic rhinitis, atopic dermatitis, atheroma/atheroschleosis, organ transplant rejection, and rheumatoid arthritis. Accordingly, they are useful for the treatment of asthma, allergic rhinitis, atopic dermatitis, atheroma/atheroschleosis, organ transplant rejection, and rheumatoid arthritis. For instance, several of the chemokine receptor modulators of the invention such as the RANTES and SDF-1α or SDF-1β antagonists also have been shown to inhibit HIV-1 infection, and antagonists (e.g., vMIP-II) can be used for the same purpose. Thus, the RANTES, or SDF-1α or SDF-1β antagonists and the vMIP-II analogues of the invention can be used for inhibiting HIV-1 in mammals. The potential of the compounds for utility against HIV-1 is determined by the method, described in the following Examples. The potential of the compounds for utility against inflammatory effects is determined by methods well known to those skilled in the art. Moreover, it will be understood that the chemokine receptor modulators of the invention can be utilized alone, or in combination with each other, as well as in combination with other non-chemokine drugs that are synergistic in treating a given disorder.

By way of example, and not by way of limitation, the following are some specific examples of wild type chemokines molecules and their associated biological properties to illustrate the general utility of making chemokine receptor modulators of these molecules. For instance, SCM-1 is a C-Chemokine expressed in spleen. It is substantially related to the CC and CXC-Chemokines, with a primary difference being that it only has the second and fourth of the four cysteines conserved in these proteins (Yoshida et al. FEBS Letters (1995) 360(2):155-159); Yoshida et al. J. Biol. Chem. (1998) 273(26):16551-16554). In humans, there are two highly homologous SCM-1 proteins, SCM-1α and SCM-1β, which differ by two amino acid substitutions. SCM-1 is found to be about 60% identical with lymphotactin, a murine lymphocyte-specific chemokine. SCM-1 and lymphotactin may thus represent the human and murine prototypes of C-Chemokines or Gamma-Chemokines. Both SCM-1 molecules specifically induce migration in murine L1.2 cells engineered to express the orphan receptor, GPR5, which is expressed primarily in placenta, and weakly in spleen and thymus among various human tissues. Accordingly, antagonists of SCM-1 find use in blocking the normal function of GPR4.

As another example, the soluble from of Fractalkine, a 76 amino acid CXXXC-chemokine, is a potent chemoattractant for T-cells and monocytes but not for neutrophils. Fractalkine is increased markedly after stimulation with TNF or IL1. The human receptor for Fractalkine is designated CX3CR1. The receptor mediates both the adhesive and migratory functions of Fractalkine. The human receptor is expressed in neutrophils, monocytes, T-lymphocytes, and several solid organs, including brain. The receptor has been shown to function with CD4 as a coreceptor for the envelope protein from a primary isolate of HIV-1. A cell-cell fusion assay demonstrates that Fractalkine potently and specifically inhibits fusion. (See, e.g., Bazan et al Nature (1997) 385(6617):640-644; Combadiere et al. J. Biol. Chem. (1998) 273(37):23799-23804; Rossi et al. Genomics (1998) 47(2):163-170; and Faure et al. Science (2000) 287:2274-2277). It is therefore apparent that antagonists of Fractalkine can find use in the treatment of various arthritic disorders involving the TNF or IL1 pathway, such as arthritis, as well as finding use as a blocker of HIV infection.

Eotaxin is an additional example. This protein is 74 amino acids in length, and is classified as a CC-Chemokine due to its characteristic cysteine pattern. It has been found in the bronchoalveolar lavage of guinea pigs used as a model of allergic inflammation, and implicated in asthma-related disorders. Eotaxin induces substantial eosinophil accumulation at a 1-2 pM dose in the skin without significantly affecting the accumulation of neutrophils. Eotaxin is a potent stimulator of both guinea pig and human eosinophils in vitro. The factor appears to share a binding site with RANTES on guinea pig eosinophils. Eotaxin induces a calcium flux response in normal human eosinophils, but not in neutrophils or monocytes. The response cannot be desensitized by pretreatment of eosinophils with other CC-Chemokines. In basophils Eotaxin induces higher levels of chemotactic response than RANTES, but it only has a marginal effect on either histamine release or leukotriene C4 generation. It also may play a role in chemotaxis of B-cell lymphoma cells. The primary receptor for Eotaxin is CCR3. (See, e.g., Bartels et al., Biochem. Biophys. Res. Comm. (1996) 225(3):1045-51); Jose et al., J. Exp. Med. (1994) 179:881-887); Ponath et al., J. Clin. Investigation (1996) 97(3):604-612); Ponath et al., J. Exp. Med. (1996) 183(6):2437-2448); Yamada et al., Biochem. Biophys. Res. Comm. (1997) 231(2):365-368). Accordingly, antagonists of Eotaxin can be used as potent modulators of asthma and other eosinophil related allergic disorders.

RANTES is another example of a target chemokine for which antagonists are of particular interest. It is a CC-Chemokine involved in many disorders ranging from inflammation, organ rejection to HIV infection. The synthesis of RANTES is induced by TNF-alpha and IL1-alpha, but not by TGF-beta, IFN-gamma and IL6. RANTES is produced by circulating T-cells and T-cell clones in culture but not by any T-cell lines tested so far. The expression of RANTES is inhibited following stimulation of T-lymphocytes. RANTES is chemotactic for T-cells, human eosinophils and basophils and plays an active role in recruiting leukocytes into inflammatory sites. RANTES also activates eosinophils to release, for example, eosinophilic cationic protein. It changes the density of eosinophils and makes them hypodense, which is thought to represent a state of generalized cell activation and is associated most often with diseases such as asthma and allergic rhinitis. RANTES also is a potent eosinophil-specific activator of oxidative metabolism. RANTES increases the adherence of monocytes to endothelial cells. It selectively supports the migration of monocytes and T-lymphocytes expressing the cell surface markers CD4 and UCHL1. These cells are thought to be pre-stimulated helper T-cells with memory T-cell functions. RANTES activates human basophils from some select basophil donors and causes the release of histamines. On the other hand RANTES can also inhibit the release of histamines from basophils induced by several cytokines including one of the most potent histamine inducers, MCAF.

RANTES has been shown recently to exhibit biological activities other than chemotaxis. It can induce the proliferation and activation of killer cells known as CHAK (C-C-Chemokine-activated killer), which are similar to cells activated by IL2. RANTES is expressed by human synovial fibroblasts and may participate in the ongoing inflammatory process in rheumatoid arthritis. High affinity receptors for RANTES (approximately 700 binding sites/cell; Kd=700 picoM) have been identified on the human monocytic leukemia cell line THP-1, which responds to RANTES in chemotaxis and calcium mobilization assays. The chemotactic response of THP-1 cells to RANTES is markedly inhibited by pre-incubation with MCAF (monocyte chemotactic and activating factor) or MIP-1-alpha (macrophage inflammatory protein). Binding of RANTES to monocytic cells is competed for by MCAF and MIP-1-alpha. Receptors for RANTES are CCR1, CCR3 and CCR5. The clinical use and significance of antagonists of RANTES is multifold. For instance, antibodies to natural RANTES can dramatically inhibit the cellular infiltration associated with experimental mesangioproliferative nephritis. In addition, natural RANTES appears to be expressed highly in human renal allografts undergoing cellular rejection related to transplant rejection of the kidney (Pattison et al., Lancet (1994) 343(8891): 209-11 (1994). Chemically modified forms of RANTES (Aminooxypentane-RANTES or AOP-RANTES; and n-nonanoyl-RANTES or NNY-RANTES) have been shown to act as an antagonist for the CCR-5 receptor of chemokines and to have the ability to inhibit HIV-1 infection. Accordingly, the antagonist N-, C- and N-/C-terminal modified analogs of RANTES according to present invention are useful as an anti-inflammatory agent in the treatment of diseases such as asthma, allergic rhinitis, atopic dermatitis, organ transplant, atheroma/atherosclerosis and rheumatoid arthritis.

Antagonists of the chemokines SDF-1α and β are additional examples, which belong to the CXC class of chemokines. SDF-1β differs by having four additional amino acids at the C-terminus. These chemokines are more than 92% identical to their non-human counterparts. SDF-1 is expressed ubiquitously with the exception of blood cells. SDF-1 acts on lymphocytes and monocytes, but not neutrophils in vitro and is a highly potent chemoattractant for mononuclear cells in vivo. It also induces intracellular actin polymerization in lymphocytes. SDF-1 acts both in vitro and in vivo as a chemoattractant for human hematopoietic progenitor cells, giving rise to mixed types of progenitors, and more primitive types. SDF-1 also appears to be involved in ventricular septum formation. Chemotaxis of CD34+ cells is increased in response to a combination of SDF-1 and IL-3. SDF has been shown also to induce a transient elevation of cytoplasmic calcium in these cells. A primary receptor for SDF-1 is CXCR4, which also functions as a major T-lymphocyte coreceptor for HIV1. See, e.g., Aiuti et al, J. Exp. Med. (1997) 185(1):111-120 (1997); Bleul et al., J. Exp. Med. (1996) 184(3):1101-1109 (1996); Bleul et al., Nature (1996) 382(6594):829-833; D'Apuzzo et al. European J. Immunol. (1997) 27(7):1788-1793; Nagasawa et al., Nature (1996) 382:635-638); Oberlin et al., Nature (1996) 382(6594):833-835. So for instance, the SDF-1 antagonists of the present invention are useful as an anti-inflammatory agent in the treatment of diseases such as asthma, allergic rhinitis, atopic dermatitis, atheroma/atherosclerosis and rheumatoid arthritis. Moreover, the SDF-1 antagonists of the invention can be used alone or in combination with other compounds, such as the RANTES antagonist analogs of the invention, for blocking the effects of SDF-1, RANTES, MIP-1α, and/or MIP-1β in mammals with respect to the recruitment and/or activation of pro-inflammatory cells, or treating or blocking HIV-1 infection.

Accordingly, another aspect of the invention relates to pharmaceutical compositions and methods of treating a mammal in need thereof by administering therapeutically effective amounts of compounds comprising the chemokine receptor modulators of the invention, or pharmaceutically acceptable salts thereof. By “pharmaceutically acceptable salt” is intended to mean a salt that retains the biological effectiveness and properties of the polypeptides of the invention and which are not biologically or otherwise undesirable. Salts may be derived from acids or bases. Acid addition salts are derived from inorganic acids, such as hydrochloric acid, hydrobromic acid, sulfuric acid (giving the sulfate and bisulfate salts), nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, salicylic acid, p-toluenesulfonic acid, and the like. Base addition salts may be derived from inorganic bases, and include sodium, potassium, lithium, ammonium, calcium, magnesium salts, and the like. Salts derived from organic bases include those formed from primary, secondary and tertiary amines, substituted amines including naturally-occurring substituted amines, and cyclic amines, including isopropylamine, trimethylamine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, N-ethylpiperidine, and the like. Preferred organic bases are isopropylamine, diethylamine, ethanolamine, piperidine, tromethamine, and choline.

The term “treatment” as used herein covers any treatment of a disease in a mammal, particularly a human, and includes: (i) preventing the disease from occurring in a subject which may be predisposed to the disease but has not yet been diagnosed as having it; (ii) inhibiting the disease, i.e. arresting its development; or (iii) relieving the disease, i.e. causing regression of the disease.

By the term “a disease state in mammals that is prevented or alleviated by treatment with a chemokine receptor modulator” as used herein is intended to cover all disease states which are generally acknowledged in the art to be usefully treated with chemokine receptor modulators in general, and those disease states which have been found to be usefully prevented or alleviated by treatment with the specific compounds of the invention. These include, by way of illustration and not limitation, asthma, allergic rhinitis, atopic dermatitis, viral diseases, atheroma/atheroschleosis, rheumatoid arthritis and organ transplant rejection.

As used herein, the term “therapeutically effective amount” refers to that amount of a chemokine receptor modulators of the invention which, when administered to a mammal in need thereof, is sufficient to effect treatment (as defined above), for example, as an anti-inflammatory agent, anti-asthmatic agent, an immunosuppressive agent, or anti-autoimmune disease agent to inhibit viral infection in mammals. The amount that constitutes a “therapeutically effective amount” will vary depending on the chemokine derivative, the condition or disease and its severity, and the mammal to be treated, its weight, age, etc., but may be determined routinely by one of ordinary skill in the art with regard to contemporary knowledge and to this disclosure. As used herein, the term “q.s.” means adding a quantity sufficient to achieve a stated function, e.g., to bring a solution to a desired volume (e.g., 100 mL).

The chemokine receptor modulators of this invention and their pharmaceutically acceptable salts, i.e., the active ingredient, are administered at a therapeutically effective dosage, i.e., that amount which, when administered to a mammal in need thereof, is sufficient to effect treatment, as described above. Administration of the chemokine receptor modulators described herein can be via any of the accepted modes of administration for agents that serve similar utilities. As used herein, the terms “chemokine receptor modulators of this invention”, “[pharmaceutically acceptable salts of] the polypeptides of the invention” and “active ingredient” are used interchangeably.

The level of the chemokine receptor modulator(s) in a formulation can vary within the full range employed by those skilled in the art, e.g., from about 0.01 percent weight (% w) to about 99.99%/w of the chemokine receptor modulator based on the total formulation and about 0.01% w to 99.99% w excipient. More typically, the chemokine receptor modulator(s) will be present at a level of about 0.5% w to about 80% w.

While human dosage levels have yet to be optimized for the chemokine receptor modulators of the invention, generally, a daily dose is from about 0.05 to 25 mg per kilogram body weight per day, and most preferably about 0.01 to 10 mg per kilogram body weight per day. Thus, for administration to a 70 kg person, the dosage range would be about 0.07 mg to 3.5 g per day, preferably about 3.5 mg to 1.75 g per day, and most preferably about 0.7 mg to 0.7 g per day. The amount of antagonist administered will, of course, be dependent on the subject and the disease state targeted for prevention or alleviation, the nature or severity of the affliction, the manner and schedule of administration and the judgment of the prescribing physician. Such use optimization is well within the ambit of those of ordinary skill in the art.

Administration can be via any accepted systemic or local route, for example, via parenteral, oral (particularly for infant formulations), intravenous, nasal, bronchial inhalation (i.e., aerosol formulation), transdermal or topical routes, in the form of solid, semi-solid or liquid or aerosol dosage forms, such as, for example, tablets, pills, capsules, powders, liquids, solutions, emulsion, injectables, suspensions, suppositories, aerosols or the like. The chemokine receptor modulators of the invention can also be administered in sustained or controlled release dosage forms, including depot injections, osmotic pumps, pills, transdermal (including electrotransport) patches, and the like, for the prolonged administration of the polypeptide at a predetermined rate, preferably in unit dosage forms suitable for single administration of precise dosages. The compositions will include a conventional pharmaceutical carrier or excipient and a chemokine receptor modulators of the invention and, in addition, may include other medicinal agents, pharmaceutical agents, carriers, adjuvants, etc. Carriers can be selected from the various oils, including those of petroleum, animal, vegetable or synthetic origin, for example, peanut oil, soybean oil, mineral oil, sesame oil, and the like. Water, saline, aqueous dextrose, and glycols are preferred liquid carriers, particularly for injectable solutions. Suitable pharmaceutical carriers include starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the like. Other suitable pharmaceutical carriers and their formulations are described in “Remington's Pharmaceutical Sciences” by E. W. Martin.

If desired, the pharmaceutical composition to be administered may also contain minor amounts of non-toxic auxiliary substances such as wetting or emulsifying agents, pH buffering agents and the like, such as for example, sodium acetate, sorbitan monolaurate, triethanolamine oleate, etc.

Although more of the active ingredient may be required, oral administration can be used to deliver the chemokine receptor modulators of the invention using a convenient daily dosage regimen, which can be adjusted according to the degree of prevention desired or in the alleviation of the affliction. For such oral administration, a pharmaceutically acceptable, non-toxic composition is formed by the incorporation of any of the normally employed excipients, such as, for example, pharmaceutical grades of mannitol, lactose, starch, povidone, magnesium stearate, sodium saccharine, talcum, cellulose, croscarmellose sodium, glucose, gelatin, sucrose, magnesium carbonate, and the like. Such compositions take the form of solutions, suspensions, dispersible tablets, pills, capsules, powders, sustained release formulations and the like. Oral formulations are particularly suited for treatment of gastrointestinal disorders. Oral bioavailablity for general systemic purposes can be adjusted by utilizing excipients that improve uptake to systemic circulation, such as formulation comprising acetylated amino acids. See, e.g., U.S. Pat. Nos. 5,935,601 and 5,629,020.

The compositions may take the form of a capsule, pill or tablet and thus the composition will contain, along with the active ingredient, a diluent such as lactose, sucrose, dicalcium phosphate, and the like; a disintegrant such as croscarmellose sodium, starch or derivatives thereof; a lubricant such as magnesium stearate and the like; and a binder such as a starch, polyvinylpyrrolidone, gum acacia, gelatin, cellulose and derivatives thereof, and the like.

Liquid pharmaceutically administrable compositions can, for example, be prepared by dissolving, dispersing, etc. a chemokine receptor modulator of the invention (about 0.5% to about 20%) and optional pharmaceutical adjuvants in a carrier, such as, for example, water, saline, aqueous dextrose, glycerol, glycols, ethanol, preservatives and the like, to thereby form a solution or suspension. If desired, the pharmaceutical composition to be administered may also contain minor amounts of nontoxic auxiliary substances such as wetting agents, suspending agents, emulsifying agents, or solubilizing agents, pH buffering agents and the like, for example, sodium acetate, sodium citrate, cyclodextrine derivatives, polyoxyethylene, sorbitan monolaurate or stearate, etc. Actual methods of preparing such dosage forms are known, or will be apparent, to those skilled in this art; for example, see Remington's Pharmaceutical Sciences, Mack Publishing Company, Easton, Pa. The composition or formulation to be administered will, in any event, contain a quantity of the active ingredient in an amount effective to prevent or alleviate the symptoms of the subject being treated. For oral administration to infants, a liquid formulation (such as a syrup or suspension) is preferred.

For a solid dosage form containing liquid, the solution or suspension, in for example propylene carbonate, vegetable oils or triglycerides, is preferably encapsulated in a gelatin capsule. For a liquid dosage form, the solution, e.g. in a polyethylene glycol, may be diluted with a sufficient quantity of a pharmaceutically acceptable liquid carrier, e.g. water, to be easily measured for administration.

Alternatively, liquid or semi-solid oral formulations may be prepared by dissolving or dispersing the active ingredient in vegetable oils, glycols, triglycerides, propylene glycol esters (e.g. propylene carbonate) and the like, and encapsulating these solutions or suspensions in hard or soft gelatin capsule shells.

In applying the chemokine receptor modulators of this invention to treatment of the above conditions, administration of the active ingredients described herein are preferably administered parenterally. Parenteral administration is generally characterized by injection, either subcutaneously, intramuscularly or intravenously, and can include intradermal or intraperitoneal injections as well as intrasternal injection or infusion techniques. Injectables can be prepared in conventional forms, either as liquid solutions or suspensions, solid forms suitable for solution or suspension in liquid prior to injection, as emulsions or in biocompatible polymer-based microspheres (e.g., liposomes, polyethylene glycol derivatives, poly(D,C)lactide and the like). Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents, solubility enhancers, protein carriers and the like, such as for example, sodium acetate, polyoxyethylene, sorbitan monolaurate, triethanolamine oleate, cyclodextrins, serum albumin etc.

The chemokine receptor modulators of the present invention can be administered parenterally, for example, by dissolving the chemokine receptor modulator in a suitable solvent (such as water or saline) or incorporation in a liposomal formulation followed, by dispersal into an acceptable infusion fluid. A typical daily dose of a polypeptide of the invention can be administered by one infusion, or by a series of infusions spaced over periodic intervals. For parenteral administration there are especially suitable aqueous solutions of an active ingredient in water-soluble form, for example in the form of a water-soluble salt, or aqueous injection suspensions that contain viscosity-increasing substances, for example sodium carboxymethylcellulose, sorbitol and/or dextran, and, if desired, stabilizers. The active ingredient, optionally together with excipients, can also be in the form of a lyophilisate and can be made into a solution prior to parenteral administration by the addition of suitable solvents.

A more recently devised approach for parenteral administration employs the implantation of a slow-release or sustained-release system, such that a constant level of dosage is maintained. See, e.g., U.S. Pat. Nos. 3,710,795, 5,714,166 and 5,041,292, which are hereby incorporated by reference.

The percentage of the active ingredient contained in such parental compositions is highly dependent on the specific nature thereof, as well as the activity of the polypeptide and the needs of the subject. However, percentages of active ingredient of 0.01% to 10% in solution are employable, and will be higher if the composition is a solid which will be subsequently diluted to the above percentages. Preferably the composition will comprise 0.02-8% of the active ingredient in solution.

Another method of administering the chemokine receptor modulators of the invention utilizes both a bolus injection and a continuous infusion. This is a particularly preferred method when the therapeutic treatment is for the prevention of HIV-1 infection.

Aerosol administration is an effective means for delivering the chemokine receptor modulators of the invention directly to the respiratory tract. Some of the advantages of this method are: 1) it circumvents the effects of enzymatic degradation, poor absorption from the gastrointestinal tract, or loss of the therapeutic agent due to the hepatic first-pass effect; 2) it administers active ingredients which would otherwise fail to reach their target sites in the respiratory tract due to their molecular size, charge or affinity to extra-pulmonary sites; 3) it provides for fast absorption into the body via the alveoli of the lungs; and 4) it avoids exposing other organ systems to the active ingredient, which is important where exposure might cause undesirable side effects. For these reasons, aerosol administration is particularly advantageous for treatment of asthma, local infections of the lung, and other diseases or disease conditions of the lung and respiratory tract.

There are three types of pharmaceutical inhalation devices, nebulizers inhalers, metered-dose inhalers and dry powder inhalers. Nebulizer devices produce a stream of high velocity air that causes the chemokine derivative (which has been formulated in a liquid form) to spray as a mist which is carried into the patient's respiratory tract. Metered-dose inhalers typically have the formulation packaged with a compressed gas and, upon actuation, discharge a measured amount of the polypeptide by compressed gas, thus affording a reliable method of administering a set amount of agent. Dry powder inhalers administer the polypeptide in the form of a free flowing powder that can be dispersed in the patient's air-stream during breathing by the device. In order to achieve a free flowing powder, the chemokine derivative is formulated with an excipient, such as lactose. A measured amount of the chemokine derivative is stored in a capsule form and is dispensed to the patient with each actuation. All of the above methods can be used for administering the present invention.

Pharmaceutical formulations based on liposomes are also suitable for use with the chemokine receptor modulators of this invention. See, e.g., U.S. Pat. Nos. 5,631,018, 5,723,147, and 5,766,627. The benefits of liposomes are believed to be related to favorable changes in tissue distribution and pharmacokinetic parameters that result from liposome entrapment of drugs, and may be applied to the polypeptides of the present invention by those skilled in the art. Controlled release liposomal liquid pharmaceutical formulations for injection or oral administration can also be used.

For systemic administration via suppository, traditional binders and carriers include, for example, polyethylene glycols or triglycerides, for example PEG 1000 (96%) and PEG 4000 (4%). Such suppositories may be formed from mixtures containing the active ingredient in the range of from about 0.5 w/w % to about 10 w/w %; preferably from about 1 w/w % to about 2 w/w %.

As described above, and further illustrated in the specific Examples that follow, the chemokine receptor modulators of the invention find use as antagonist of the naturally occurring chemokines. In particular, the chemokine receptor modulators of the invention having enhanced potency as an antagonist find use in the analysis and treatment of various disease states, such as asthma, allergic rhinitis, atopic dermatitis, organ transplant rejection, viral diseases, atheroma/atheroschleosis, rheumatoid arthritis and organ transplant rejection. The chemokine receptor modulators of the invention also can be utilized in designing and screening small molecule antagonist of their cognate receptors. For instance, the structural diversity engineered into the antagonist compounds of the invention facilitates a more rational approach in the design, screening and fine tuning of better small molecule compounds for use as medicaments in the treatment of diseases involving the natural activity of chemokine receptors.

### EXAMPLES

The following preparations and examples are given to enable those skilled in the art to more clearly understand and to practice the present invention. They should not be considered as limiting the scope of the invention, but merely as being illustrative and representative thereof.

### Abbreviations

| | |
|---|---|
| DIEA | diisopropylethyleamine |
| DMF | N,N-dimethylformamide |
| DNP | 2,4-dinitrophenyl |
| GuHCl | guanidinium hydrochloride |
| HBTU | O-(1H-benzotriazol-1-yl)-1,1,3,3- |
| | tetramethyl-uronium |
| | hexafluorophosphate |
| HF | hydrogen fluoride |
| TFA | trifluoroacetic acid |
| Aib | aminoisobutyric acid |
| Hyp | hydroxyproline |
| Tic | 1,2,3,4-tetrahydroisoquinoline-3- |
| | COOH |
| Indol | indoline-2-carboxylic acid |
| P(4,4DiF) | 4-difluoro-proline |
| Thz | L-thiazolidine-4-carboxylic acid |
| Hop | L-homoproline |
| ΔPro | 3,4-dehydro-proline |
| F(3,4-DiOH) | 3,4dihydroxyphenylalanine |
| F(3,4-DiOH, pBzl)) | pBzl,-3,4dihydroxyphenylalanine |
| p-Bz | benzophenone |
| Cha | cyclohexyl-alanine |
| βNal | 3-(2-naphtyl)-alanine |
| Chg | cyclohexyl-glycine |
| Phg | phenylglycine |
| HoF | homophenylalanine |
| F(F)₅ | pentafluorophenylalanine |
| tBuA | tert-butylalanine |
| F(4-Me) | 4-methylphenylalanine |
| tL | tert-leucine |
| CycP | 1-amino-1-cyclopentanecarboxylic |
| | acid |
| CycH | 1-amino-1-cyclohexanecarboxylic |
| | acid |
| Nle | norleucine |
| Aminooxypentane-RANTE(2-68) | AOP-RANTES |
| n-Nonanoyl-RANTES(2-68) | NNY-RANTES |

### Example 1

### General Synthesis Approach for Chemokine Receptor Modulators

Peptides for chemokine receptor modulators were made by solid-phase peptide synthesis. Solid-phase synthesis was performed on a custom-modified 430A peptide synthesizer from Applied Biosystems, using in situ neutralization/2-(1H-benzotriazol-1-yl)-1,1,1,3,3-tetramethyluronium hexa fluorophosphate activation protocols for stepwise Boc chemistry chain elongation (Schnolzer, et al., Int. J. Peptide Protein Res. (1992) 40:180-193). The N-terminal peptide fragments were synthesized on a thioester-generating resin. The resin was split after attachment of the residue preceding the position investigated (elongation from C to N terminus) and the peptide elongated manually on a 0.03 mmol scale. Each synthetic cycle consisted of Nα-Boc-removal by a 1 to 2 minute treatment with neat TFA, a 1-min DMF flow wash, a 10- to 20-minute coupling time with 1.0 mmol of preactivated Boc-amino acid in the presence of excess DIEA and a second DMF flow wash. Nα-Boc-amino acids (1.1 mmol) were preactivated for 3 minutes with 1 mmol HBTU (0.5M in DMF) in the presence of excess DIEA (3 mmol). After each manual coupling step, residual free amine was evaluated with the ninhydrin assay (Sarin, et al., Anal. Biochem. (1981) 117:147-157). The C-terminal fragment comprising amino acids were synthesized on a standard —O—CH₂-phenylacetamidomethyl resin. After chain assembly was completed, the peptides were deprotected and cleaved from the resin by treatment with anhydrous HF for 1 hour at 0° C. with 5% p-cresol as a scavenger. In all cases, the imidazole side chain DNP protecting groups remained on His residues because the DNP-removal procedure is incompatible with C-terminal thioester groups. However DNP was gradually removed by thiols during the ligation reaction, yielding unprotected His. After cleavage, both peptides were precipitated with ice cold diethylether, dissolved in aqueous acetonitrile and lyophilized. The peptides were purified by RP-HPLC with a C18-column from Waters by using linear gradients of buffer B (acetonitile/10% H₂O/0.1% trifluoroacetic acid) in buffer A (H₂O/0.1% trifluoroacetic acid) and UV detection at 214 nm. Samples were analyzed by electrospray mass spectrometry with a Platform II instrument (Micromass, Manchester, England). Peptides were utilized for ligation to generate full-length chemokine polypeptide chains using native chemical ligation (Dawson, et al., Science (1994) 266:776-779); Wilken, et al., Chem. Biol. (1999) 6:43-51; and Camarero, et al., Current Protocols in Protein Science (1999) 18.4.1-18.4.21). Folding of the polypeptide chains was accomplished in the presence of Cys-SH/(Cys-S)₂ following standard techniques (Wilken et al., Chem. Biol. (1999) 6:43-51).

### Example 2

### Synthesis of N-, C- and N-/C-terminal Analogs of NNY-RANTES, AOP-RANTES, and SDF-1

Analogs of RANTES (1-68) and SDF-1β (1-72 ) were prepared as in Example 1 and described herein to illustrate a general approach of making CC and CXC chemokine antagonists. In particular, N-terminal, C-terminal and N-/C-terminal modified RANTES analogs were based on modifications to the chemokine compound CH₃—(CH₂)₇—C(O)-RANTES (2-68), also referred to as n-nonanoyl-RANTES (2-68) or “NNY-RANTES”, and the chemokine compound CH₃—(CH₂)₄—O—N═CH—CO-RANTES (2-68), also referred to as aminooxypentane-RANTES or “AOP-RANTES”. The NNY-RANTES, AOP-RANTES and additional RANTES derivative molecules utilized for this purpose are described in WO 99/11666, which reference is incorporated herein by reference. The N-, C- and N-/C-terminal analogs of SDF-1 were constructed using the same basic design approach as for the RANTES analogs.

For the N-terminal modifications to a given target chemokine, such as the NNY and AOP modifications to RANTES, chemical variants were prepared as described above and in WO 99/11666 and Wilken et al., Chem. Biol. (1999) 6:43-51, utilizing on-resin elaboration of the N-terminal peptide segment employed for ligation to generated the pendant N-terminal modification (e.g., NNY or AOP), followed by cleavage/deprotection, purification and use of the unprotected N-terminal modified peptide α-thioester in native chemical ligation to the C-terminal peptide segment to form the full length product. Peptides were synthesized and amino acid substitutions, including amino acid derivatives, were incorporated during peptide synthesis as described in Example 1. Native chemical ligation as in Example 1 was utilized to generate the linear product, where ligation was at the Lys³¹-Cys³² site for the RANTES analogs, and for the SDF-1 analogs at the Asn³³-Cys³⁴ site. Equimolar amount of peptide fragments (2-2.5 mM) were dissolved in 6M GuHCl, 100 mM phosphate, pH 7.5, 1% benzylmercaptan, and 3% thiophenol. The reactions usually were carried out overnight. The resulting polypeptide products were purified and analyzed as described above for peptide segments. For generating the folded protein, the purified polypeptide chains of NNY-RANTES analogs (about 0.5 to 1 mg/mL) were dissolved in 2M GuHCl, 100 mM Tris, pH 8.0 containing 8 mM cysteine, 1 mM cystine and 10 mM methionine. After gentle stirring overnight, the protein solution was purified by RP-HPLC as described above. Other folding conditions were used in the case of SDF-1 analogs: SDF-1 and Met⁰-SDF-1 were oxidized at 0.5 mg/mL in 1M GuHCl, 0.1M Tris, pH_{8.5} at room temperature in the presence of air. After stirring overnight, folding was complete. AOP-, caproyl- and NNY-SDF-1 were oxidized in the same buffer but in the presence of 2M GuHCl.

For chemical conjugation of the fatty acid to a given folded protein, two basic steps were involved. First, the fatty acid was functionalized with an amino oxy group. Second, a reactive carbonyl group was introduced specifically in the carboxyl-terminal domain of the protein, a region believed not to be critical for the activity of chemokines. For this purpose, chemokine analogs targeted for C-terminal fatty acid modification were synthesized with a C-terminal Lys(Ser)Gly sequence extension. Thus, for example, NNY-RANTES (2-68) was synthesized to contain a Lys(Ser)Gly sequence extension at the C-terminus. The reactive carbonyl group was generated by NaIO₄ treatment of the refolded protein, thus allowing the site-specific attachment of the fatty acid moiety through a stable oxime bond.

For fatty acid functionalization, 0.2 mmol fatty acid (palmitate, oleate, arachidonate, cholate) was activated with equimolar amounts of DCC and HOAt in 0.5 ml of DMF/DCM mixture (1:1, v:v) and added to a 0.5 ml DMF solution of 0.25 mmol Boc-AoA-NH—(CH₂)₂—NH₂ and the apparent pH adjusted to pH.8.0 with N-ethylmorpholine. For the cholesteryl derivative, 0.2 mmol cholesteryl-chloroformate was dissolved in 0.5 ml DCM and added to an ethanolic solution of 0.25 mmol Boc-AoA-NH—(CH₂)₂—NH₂ and the apparent pH adjusted to pH 9.0 with triethylamine. After overnight incubation the volatiles were removed under vacuum and the product isolated either by flash chromatography or by preparative HPLC on a C4 column The Boc group was removed by TFA treatment and the product verified by ESI-MS.

For protein oxidation, the target protein (2 mg/mL) was dissolved in a 0.1 M sodium phosphate buffer, pH_{7.5} containing 6M guanidine chloride and methionine added to get a 100-fold molar excess of scavenger over protein. A 10-fold excess of sodium periodate was then added and the solution incubated for 10 min in the dark. The reaction was stopped by the addition of a 1000-fold molar excess ethylene glycol over periodate and the solution further incubated for 15 min at room temperature. The solution was then dialyzed against 0.1% acetic acid and finally lyophilized. For example, oxidation of the C-terminal lateral serine was shown to be almost quantitative by ESI-MS, where a mass of 8141.1±0.7 Da was obtained in the case of AOP-RANTES-K(S)G, corresponding to the loss of 31 Da to form the glyoxylyl derivative and no peak corresponding to the mass of the starting material was observed.

Conjugation of the fatty acid with the chemokine was accomplished in 0.1 M sodium acetate buffer, pH 5.3, in the presence of 0.1% sarcosyl, 20 mM methionine and a 20-fold-excess of functionalized fatty acid over the protein. After agitation for 16-20 h at 37° C., the conjugate, as an oxime bond formed between the amino-oxy group of the fatty acid and the chemokine aldehyde, was purified using reverse phase-HPLC and the product characterized by ESI-MS. For all analogs, the coupling of aminooxy-functionalized fatty acids to oxidized protein was almost quantitative as controlled by analytical HPLC.

### Example 3

### N-terminal Analogs of NNY- and AOP-RANTES

For the N-terminal RANTES derivatives, the modifications were made to one or more of the N-terminal region of amino acids corresponding to the first eight amino acid residues of NNY-RANTES (2-68) or AOP-RANTES (2-68), which first eight amino acid residues have the following sequence -PYSSDTTP-. These correspond to amino acid residues 2-9 of the 68 amino acid residue wild type RANTES polypeptide chain (i.e., RANTES (1-68)) shown in FIGS. 2A-2E, since the first residue (Ser) of naturally occurring RANTES (1-68) is replaced by the n-nonanoyl substituent in NNY-RANTES (2-68) and aminooxypentane in AOP-RANTES (2-68). So for example, a substitution in NNY-RANTES (2-68) at amino acid position 2 is indicated below by the general compound formula “NNY-P2X-RANTES (3-68)”, where NNY is n-nonanoyl, X is an amino acid substituted for the proline (P) at position 2 of NNY-RANTES (2-68), and RANTES (3-68) represents the remaining 66 amino acids of NNY-RANTES (2-68), as read in the N- to C-terminal direction. By way of another example, a substitution in NNY-RANTES (2-68) at amino acid position 3 is indicated by the general compound formula “NNY-P-Y3X-RANTES (4-68)”, where NNY is n-nonanoyl, X is an amino acid substituted for the tyrosine (Y) at position 3 of NNY-RANTES (2-68), and RANTES (4-68) represents the remaining 65 amino acids of NAY-RANTES (2-68), as read in the N- to C-terminal direction. For multiply substituted NNY-RANTES analogs, the following example of a compound formula for three substitutions in NNY-RANTES (2-68) at amino acid positions 2, 3 and 9 is indicated by the general compound formula “NNY-P2X-Y3X-SSDTT-P9X-RANTES (10-68)”, where NNY is n-nonanoyl, X is the same or different amino acid substituted for the proline (P) at position 2, tyrosine (Y) at position 3, and proline (P) 9 of NNY-RANTES (2-68), SSDTT corresponds to amino acids 4-8 of NNY-RANTES (2-68), and RANTES (10-68) represents the remaining 59 amino acids of NNY-RANTES (2-68), as read in the N- to C-terminal direction. The following are examples of the NNY-P2X-RANTES (3-68) analogs prepared.

| Compound | Number |
|---|---|
| NNY-P2Aib-RANTES (3-68) | 1 |
| NNY-P2Hyp-RANTES (3-68) | 2 |
| NNY-P2Tic-RANTES (3-68) | 3 |
| NNY-P2Indol-RANTES (3-8) | 4 |
| NNY-P2P(4,4DiF)-RANTES (3-8) | 5 |
| NNY-P2Thz-RANTES (3-68) | 6 |
| NNY-P2HoP-RANTES (3-68) | 7 |
| NNY-P2ΔPro-RANTES (3-68) | 8 |
| NNY-P2A-RANTES (3-68) | 9 |

The following are examples of the NNY-P-Y3X-RANTES (4-68) analogs prepared.

| Compound | Number |
|---|---|
| NNY-P-Y3P-RANTES (4-68) | 10 |
| NNY-P-Y3A-RANTES (4-68) | 11 |
| NNY-P-Y3L-RANTES (4-68) | 12 |
| NNY-P-Y3V-RANTES (4-68) | 13 |
| NNY-P-Y3F(3,4-DiOH)-RANTES (4-68) | 14 |
| NNY-P-Y3F(3,4-DiOH,pBzl)-RANTES (4-68) | 15 |
| NNY-P-Y3pBz-RANTES (4-68) | 16 |
| NNY-P-Y3Cha-RANTES (4-68) | 17 |
| NNY-P-Y3βNal-RANTES (4-68) | 18 |
| NNY-P-Y3Chg-RANTES (4-68) | 19 |
| NNY-P-Y3Phg-RANTES (4-68) | 20 |
| NNY-P-Y3Hof-RANTES (4-68) | 21 |
| NNY-P-Y3F(F)₅-RANTES (4-68) | 22 |
| NNY-P-Y3tbuA-RANTES (4-68) | 23 |
| NNY-P-Y3F(4-Me)-RANTES (4-68) | 24 |
| NNY-P-Y3tL-RANTES (4-68) | 25 |
| NNY-P-Y3CycP-RANTES (4-68) | 26 |
| NNY-P-Y3CycH-RANTES (4-68) | 27 |
| NNY-P-Y3Nle-RANTES (4-68) | 28 |

The following compounds are examples of the NNY-PY-S4X-RANTES (5-68) analogs prepared.

| Compound | |
|---|---|
| NNY-PY-S4A-RANTES (5-68) | 29 |
| NNY-PY-S4tbuA-RANTES (5-68) | 30 |

The following compounds are examples of the NNY-PYS-S5X-RANTES (6-68) analogs prepared.

| Compound | Number |
|---|---|
| NNY-PYS-S5tbuA-RANTES (6-68) | 31 |

The following compounds are examples of the NNY-PYSS-D6X-RANTES (7-68) analogs prepared.

| Compound | Number |
|---|---|
| NNY-PYSS-D6tbuA-RANTES (7-68) | 32 |

The following compounds are examples of the NNY-PYSSD-T7X-RANTES (8-68) analogs prepared.

| Compound | Number |
|---|---|
| NNY-PYSSD-T7tbuA-RANTES (8-68) | 33 |

The following compounds are examples of the NNY-PYSSDT-T8X-RANTES (9-68) analogs prepared.

| Compound | Number |
|---|---|
| NNY-PYSSDT-T8tBuA-RANTES (9-68) | 34 |

The following compounds are examples of the NNY-PYSSDTT-P9X-RANTES analogs prepared.

| Compound | Number |
|---|---|
| NNY-PYSSDTT-P9Hyp-RANTES (10-68) | 35 |
| NNY-PYSSDTT-P9Aib-RANTES (10-68) | 36 |
| NNY-PYSSDTT-P9ΔPro-RANTES (10-68) | 37 |
| NNY-PYSSDTT-P9Thz-RANTES (10-68) | 38 |

The following compounds are examples of the double substituted analogs NNY-P2X-Y3X-RANTES (4-68), and triple substituted analogs NNY-P2X-Y3X-SSDTT-P9X-RANTES (10-68) prepared.

| Compound | Number |
|---|---|
| NNY-P2Hyp-Y3tButA-RANTES (4-68) | 39 |
| NNY-P2Thz-Y3tButA-RANTES (4-68) | 40 |
| NNY-P2Hyp-Y3Chg-RANTES (4-68) | 41 |
| NNY-P2Thz-Y3Chg-RANTES (4-68) | 42 |
| NNY-P2Thz-Y3Chg-SSDTT-P9Aib-RANTES (10-68) | 43 |

### Example 4

### N-terminal, N-loop Analogs of NNY-RANTES

The following compounds are intended to be illustrative of additional NNY-substituted-RANTES analogs in which the N-loop (residues 12-20 of RANTES) is modified to increase potency towards CCR5 without affecting signal transduction via CCR1 and CCR3.

For the N-terminal, N-loop RANTES analogs, the N-loop modifications were made to NNY-RANTES (2-68), where the N-loop corresponds to amino acids 12-20. The N-loop of RANTES has the amino acid sequence -FAYIARPLP- (SEQ ID NO:2). So for example, a substitution in NNY-RANTES (2-68) at amino acid position 12 has the general compound formula “NNY-PYSSDTTPCC-F12pBz-RANTES (13-68)”, where NNY is n-nonanoyl, PYSSDTTPCC corresponds to amino acids 2-11 of RANTES (1-68), F12pBz indicates substitution of the amino acid derivative pBZ for the phenylalanine (F) at position 12 of RANTES (1-68), and RANTES (13-68) represents the remaining amino acid residues 13-68 of RANTES (1-68), as read in the N- to C-terminal direction.

| Compound | Number |
|---|---|
| NNY-PYSSDTTPCC-F12pBz-RANTES (13-68) | 44 |
| NNY-PYSSDTTPCC-F12Y-RANTES (13-68) | 45 |
| NNY-PYSSDTTPCC-F12F(4-Me)-RANTES (13-68) | 46 |
| NNY-PYSSDTTPCC-F12(4-F)-RANTES (13-68) | 47 |
| NNY-PYSSDTTPCCF-A13R-RANTES (14-68) | 48 |
| NNY-PYSSDTTPCCF-A13S-RANTES (14-68) | 49 |
| NNY-PYSSDTTPCCFA-Y14F-RANTES (15-68) | 50 |
| NNY-PYSSDTTPCCFA-Y14Cha-RANTES (15-68) | 51 |
| NNY-PYSSDTTPCCFAY-I15tBuA-RANTES (16-68) | 52 |
| NNY-PYSSDTTPCCFAY-I15S-RANTES (16-68) | 53 |
| NNY-PYSSDTTPCCFAYI-A16S-RANTES (17-68) | 54 |
| NNY-PYSSDTTPCCFAYA-R17A-RANTES (18-68) | 55 |
| NNY-PYSSDTTPCCFAYA-R17H-RANTES (18-68) | 56 |
| NNY-PYSSDTTPCCFAYAR-P18Thz-RANTES (19-68) | 57 |
| NNY-PYSSDTTPCCFAYARP-L19I-RANTES (20-68) | 58 |
| NNY-PYSSDTTPCCFAYARP-L19Cha-RANTES (20-68) | 59 |
| NNY-PYSSDTTPCCFAYARPL-P20Thz-RANTES (21-68) | 60 |

### Example 5

### N-terminal RANTES Analogs of NNY-RANTES

The following compounds are intended to be illustrative of additional NNY-substituted-RANTES analogs in which a different aliphatic chain was employed in lieu of the NNY substituent.

| Compound | Number |
|---|---|
| CH2═CH—CH2—CH2—CH2—CH2—CH2—CH2—CO- | 61 |
| RANTES (2-68) | |
| Nle-Met-RANTES (1-68) | 62 |
| Dodecanoyl-RANTES (3-68) | 63 |
| Lauryl-Hyp-RANTES (3-68) | 64 |
| Myristoyl-RANTES (4-68) | 65 |
| Dodecanoyl-Hyp-RANTES (4-68) | 66 |

### Example 6

### C-terminal and N/C-terminal Analogs of NNY- and AOP-RANTES

AOP- and NNY-RANTES having a Lys-Gly C-terminal extension, with the epsilon amino group of the Lys acylated by a serine residue were prepared. These derivatives were conjugated, after periodate oxidation of the serine extension, with aminooxyacetyl-functionalized compounds including fluorophores (FITC, NBD, Cy-5 and BODIPY-FI) or lipids. These C-terminally labeled chemokines retain their biological properties and introduction of a aliphatic moiety as like as CH₃—(CH₂)₁₄—CONH—(CH₂)₂—NHCO—CH₂—O—NH₂ was shown to improve the potency of the chemokine. In order to find out the most effective compound, different fatty acids and lipids were functionalized with an aminooxy group by coupling with Boc-AoA-NH—(CH₂)₂—NH₂, followed by Boc removal laurate, palmitate, oeate, eicosanoate, cholic acid, and cholesteryl-chloroformate. One or more of these derivatives were conjugated to oxidized NNY-RANTES-K(S)G or AOP-RANTES-K(S)G, where the AOP analogs are exemplified below:

| Compound | Number |
|---|---|
| AOP-RANTES-K(lauryl)-G | 67 |
| where “(lauryl)” is an abbreviation | |
| for gloxylyl=AoA-ethylene diamine- | |
| laurate and so on | |
| AOP-RANTES-K(palmitoyl)-G | 68 |
| AOP-RANTES-K(eicosanoyl)-G | 69 |
| AOP-RANTES-K(oleoyl)-G | 70 |
| AOP-RANTES-K(cholyl)-G | 71 |
| AOP-RANTES-K(cholesteryl)-G | 72 |

Chemical variants of the lipidic moiety were also prepared by another strategy. Such compounds were synthesized by on-resin elaboration of the C-terminal segment by attachment of the fatty acid to the Fmoc-deprotected Boc-peptide-Lys-Gly-resin, prior to cleavage, purification and use in chemical ligation to form the full length polypeptide.

In designing these compounds, there were two main reasons that the lipid coupling was utilized. First, there is now more and more evidence that the anti HIV-1 inhibitory activity of the RANTES compounds is related to the ability to down-regulate the receptor. This means that once internalized the ligand-receptor complex which should be normally dissociated in early endosomes with recycling of the receptor could also interact with the plasma membrane or some cytoplasmic fatty acid binding proteins. Accordingly, lipid modification of the ligand may retarget the complex to a specific intracellular subdomain simply through interactions and thus delaying the recycling of the receptor. Several recent papers dealing with intracellular protein trafficking support the idea that acylation is a common mechanism of increasing the affinity of proteins for detergent resistant membranes and may be the primary targeting mechanism for proteins without membrane spans (See, e.g., Melkonian et al., J. Biol.Chem. (1999) 274:3910-3917; Zlatkine et al., J. Cell Sci. (1997) 110:673-679; Zhan et al. Cancer Immunol. Immunother. (1998) 46:55-60). Second, the modification also was carried out to change the pharmacokinetic properties of the compounds. Several recent papers support this concept (see, e.g., Honeycutt et al. Pharm.Res. (1996) 13:1373-1377; Kurtzhals et al. J. Pharm. Sci. (1997) 86:1365-1368; Markussen et al. Diabetologia (1996) 39:281-288).

As demonstrated in the Examples that follow, the enhancement of activity was surprising and unexpected, since the modification was intended to change pharmacokinetics. An expected would have been that the activity decreased, but the hoped-for improvement in pharmacokinetics would have given an acceptable trade-off.

### Example 7

### N-terminal Analogs of SDF-1

The following N-terminal SDF-1 (1-72) derivatives were prepared to illustrate a general approach of making CXC chemokine receptor modulator. By way of example, the N-terminus of SDF-1 was modified to generate compounds having aliphatic chain at the N-terminus. Compounds that further include an amino acid derivative at the N-terminal region, and/or an aliphatic chain at the C-terminal region are prepared as described above for the RANTES compounds. In particular, suitable N-terminal substituents were prepared and tested that included, by way of illustration and not limitation Lys, Met-Lys, caproyl-Lys, CH₃—(CH₂)₇—C(O) and CH₃—(CH₂)₄—O—NH-glyoxylyl. The following compounds are examples of some of the SDF-1 analogs prepared.

| Compound | Number |
|---|---|
| Lys-SDF-1 (2-72) | 73 |
| Met-Lys-SDF-1 (2-72) | 74 |
| Caproyl-Lys-SDF-1 (2-72) | 75 |
| NNY-SDF-1 (2-72) | 76 |
| AOP-glyoxylyl-SDF-1 (2-72) | 77 |

### Example 8

### Screening Assays

Several of the RANTES and SDF analogs prepared in Examples 3-7 and others were screened for inhibitor activity, using an HIV-based assay to characterize the blocking function for this particular indication for which RANTES and SDF-1 find use. In general, the compounds were passed through a preliminary screen for their ability to inhibit HIV envelope-mediated cell fusion. The most promising of these compounds were subsequently tested for their ability to inhibit cell-free viral infection of a target cell line. These assays were chosen since the cell fusion assay and the in vitro cell-free viral infection assay have been found to be useful indicators of potency in vivo, as determined in the SCID mouse model (Mosier et al., J. Virol. (1999) 73:3544-3550). Moreover, since the increase in anti-viral potency of NNY-RANTES over AOP-RANTES has been found to be due to factors other than an increase in affinity for CCR5, the compounds were evaluated in terms of activity in the cell fusion assay, rather than affinity for CCR5.

### Example 9

### Envelope-Mediated Cell Fusion Assays

The ability of a given panel of compounds of Examples 3-7 to inhibit CCR5-dependent cell fusion was determined using cells engineered to viral envelop proteins fusing with cells bearing CD4 and CCR5 and containing a reporter system. CCR5-tropic viral envelope-mediated cell fusion assays were carried out essentially as described in Simmons et al. (Science (1997) 276:276-279) using the cell lines HeLa-P5L and HeLa-Env-ADA, both of which were kindly provided by the laboratory of M. Alizon (Paris). Briefly, HeLa-P5L cells were seeded in 96-well plates (10⁴ cells per well in 100 μl). Twenty-four hours later medium was removed and medium containing 10⁴ HeLa-Env-ADA cells per well plus chemokines was added (200 μl final volume). After a further twenty-four hours, cells were washed once in PBS and lysed in 50 μl PBS/0.5% NP-40 for 15 min at room temperature. Lysates were assayed for for P-galactosidase activity by the addition of 50 μl 2×CPRG substrate (16 mM chlorophenol red-β-D-galactopyranoside; 120 mM Na₂HPO₄, 80 mM NaH₂PO₄, 20 mM KCl, 20 mM MgSO₄, and 10 mM β-mercaptoethanol) followed by incubation for 1-2 hours in the dark at room temperature. The absorbance at 575 nm was then read on a Labsystems microplate reader. From these values, percentage inhibition [100×(OD₍ₜₑₛₜ₎−OD_{(negative control)})/OD_{(positive control)}−OD_{(negative control)})] was calculated at each inhibitor concentration. A plot of percentage fusion inhibition against inhibitor concentration allowed the calculation of IC₅₀ values for each compound.

Significantly, a majority of the compounds tested exhibited greater potency relative to wild type RANTES. Results for selected RANTES inhibitor analogs are shown in Table 1 below.

TABLE 1
Cell-Fusion Screen

| | |
|---|---|
| Compound Number | Mean Relative Potency |

N-terminal modified NNY-RANTES

| | |
|---|---|
| 19 | 7 |
| 23 | 7 |
| 40 | 4 |
| 42 | 2 |
| NNY-RANTES (control) | 18-25 |

N-loop modified NNY RANTES (2-68)

| | |
|---|---|
| 54 | 15 |
| 57 | 15 |
| 58 | 13 |
| 59 | 14 |
| NNY-RANTES (control) | 18-25 |

C-Terminal modified AOP-RANTES

| | |
|---|---|
| 68 | 45 |
| AOP-RANTES (Control) | 100 |

In Table 1, for the mean relative potencies, absolute values for IC₅₀s in the fusion assay vary across experiments performed on different days, although rank orders of activity remain constant. In order to normalize results, AOP-RANTES was used as a control in each experiment. So the IC₅₀s in each experiment were expressed relative to that of AOP-RANTES, which was given an arbitrary value of 100. Although most all of the compounds tested exhibited greater potency relative to wild type RANTES, potencies of certain compounds, such as compound numbers 19, 23, 40 and 42, were such that the more than 50% inhibition was obtained even at the lowest dilution in the series.

### Example 10

### Cell-free Viral Infection Assays

The cell-free viral infection assays were carried out in the same way as the envelope-mediated cell fusion assay, except that in this case the envelope cell line was replaced by live R5-tropic virus. HEK293-CCR5 cells (7, kindly provided by T. Schwartz, Copenhagen) were seeded into 24 well plates (1.2×10⁵ cells/well). After overnight incubation, competition binding was performed on whole cells for 3 h at 4° C. using 12 pM [¹²⁵]MIP-1-α (Amersham) plus variable amounts of unlabelled ligand in 0.5 ml of ‘Binding Buffer’ (50 mM HEPES, pH 7.4, supplemented with 1 mM CaCl₂, 5 mM MgCl₂, and 0.5% (w/v) bovine serum albumin). After incubation, cells were washed rapidly four times in ice cold Binding Buffer supplemented with 0.5 M NaCl. Cells were lysed in 1 ml 3 M Acetic Acid, 8 M Urea and 2% NP-40. Lysed material was counted for 1 minute using a Beckman Gamma 4000 scintillation counter. Determinations were made in duplicate and IC50 values were derived from monophasic concentration inhibition curves fitted using Prism software. Table 2 illustrates the increase in potency over NNY-RANTES shown in the preliminary screen by compound numbers 19 and 23.

TABLE 2
In Vitro Infectivity Data For Selected Compounds
From Cell-Free Viral Infection Assay

| | AOP- | NNY- | Compound | Compound |
|---|---|---|---|---|
| | RANTES | RANTES | 23 | 19 |
| Experimental | 140 | 32 | 17 | 15 |
| IC₅₀ | 47 | 8 | 3.8 | 34 |
| Infectivity | 260 | 26 | 28 | 9.9 |
| Results | 135 | 30 | 14 | 12 |
| Average | 145 pM | 24 pM | 14 pM | 12 pM |
| Infectivity | | | | |
| IC₅₀ | | | | |
| Cell-Fusion | 480 pM | 97 pM | 38 pM | 26 pM |
| Result For | | | | |
| Comparison | | | | |

### Example 11

### Combination Treatment With Anti-CCR5 and Anti-CXCR4 Compounds

The following example illustrates the protective effects of employing an anti-CCR5 (e.g., NNY-RANTES) and an anti-CXCR4 (e.g., SDF-1 antagonist or AMD 3100) in combination for blocking HIV infection, and blocking the potential conversion of R5 strains of HIV to X4 strains. A SCID mouse model was utilized for the purpose. In particular, the protective effects of NNY-RANTES and AMD 3100 (a small organic molecule anti-X4 agent) were tested in SCID mice, repopulated with human peripheral blood leukocytes and challenged with HIV-1 following the methods described in Mosier, Adv. Immunol. (1996) 63:79-125; Picchio, et al., J. Virol. (1997) 71:7124-7127; Picchio, et al., J. Virol. (1998) 72:2002-2009; and Offord et al., WO 99/11666. NNY-RANTES was administered as in Table X, and AMD 3100 used as a 200 mg/ml solution. Challenge was with an R5 HIV virus except for the AMD 3100 group alone. No escape mutants were observed in the combination therapy, and all of the appropriately treated mice remained virus free throughout the experiment. This indicates that the N-, C- and N-/C-terminal RANTES derivatives of the invention can be used in combination with anti-X4 strain compounds such as AMD 3100 or SDF-1 antagonist, such as those described herein, for blocking HIV infection in mammals.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

What is claimed is:

1. A chemokine receptor modulator comprising a chemokine polypeptide chain modified at its N-terminus with an aliphatic chain and one or more amino acid derivatives.

2. The chemokine receptor modulator of claim 1, wherein said chemokine polypeptide chain comprises an amino acid sequence that is substantially homologous to the amino acid sequence of a naturally occurring wild type chemokine.

3. The chemokine receptor modulator of claim 2, wherein said naturally occurring wild type chemokine is a CC chemokine.

4. The chemokine receptor modulator of claim 2, wherein said naturally occurring wild type chemokine is a CXC Chemokine.

5. The chemokine receptor modulator of claim 1, wherein said N-terminus comprises amino acids of said chemokine polypeptide chain that are N-terminal to the first disulfide-forming cysteine of said chemokine polypeptide chain.

6. The chemokine receptor modulator of claim 1, wherein said aliphatic chain is a hydrocarbon chain comprising 5 to 26 carbons.

7. The chemokine receptor modulator of claim 1, wherein said amino acid derivative has the formula —(N-CnR-CO)—, where n is 1-22, R is hydrogen, alkyl or aromatic, and where N and Cn, N and R, or Cn and R can form a cyclic structure.

8. A chemokine receptor modulator comprising a chemokine polypeptide chain modified at its C-terminus with an aliphatic chain or polycyclic.

9. The chemokine receptor modulator of claim 8, wherein said aliphatic chain comprises 5 to 22 carbons.

10. The chemokine receptor modulator of claim 9, wherein said aliphatic chain or polycyclic is a lipid.

12. A chemokine receptor modulator comprising a chemokine polypeptide chain modified at its N-terminus with an aliphatic chain and one or more amino acid derivatives, and at its C-terminus with an aliphatic chain or polycyclic.

13. A pharmaceutical composition comprising a chemokine receptor modulator, wherein said chemokine receptor modulator comprises a chemokine polypeptide chain modified at its N-terminus with an aliphatic chain and one or more amino acid derivatives, or a pharmaceutically acceptable salt thereof.

14. The pharmaceutical composition of claim 13, wherein said composition is in admixture with one or more pharmaceutically acceptable excipients.

15. A pharmaceutical composition comprising a chemokine receptor modulator, wherein said chemokine receptor modulator comprises a chemokine polypeptide chain modified at its C-terminus with an aliphatic chain or polycyclic, or a pharmaceutically acceptable salt thereof.

16. A pharmaceutical composition comprising the chemokine receptor modulator of claim 15 or a pharmaceutically acceptable salt thereof.

17. The pharmaceutical composition of claim 16, wherein said composition is in admixture with one or more pharmaceutically acceptable excipients.

18. A pharmaceutical composition comprising a chemokine receptor modulator comprising a chemokine polypeptide chain modified at its N-terminus with an aliphatic chain and one or more amino acid derivatives, and at its C-terminus with an aliphatic chain or polycyclic, or a pharmaceutically acceptable salt thereof.

19. The pharmaceutical composition of claim 18, wherein said composition is in admixture with one or more pharmaceutically acceptable excipients.

20. A method of treating a disease state in mammals that is alleviated by treatment with a chemokine receptor modulator, which method comprises administering to a mammal in need of such a treatment a therapeutically effective amount of a chemokine receptor modulator, wherein said chemokine receptor modulator comprises a chemokine polypeptide chain (A) modified at its N-terminus with an aliphatic chain and one or more amino acid derivatives, (B) modified at its C-terminus with an aliphatic chain or polycyclic, or (C) modified at its N-terminus with an aliphatic chain and one or more amino acid derivatives, and at its C-terminus with an aliphatic chain or polycyclic.

21. The method of claim 20, wherein the disease state is an inflammatory disease.

22. The method of claim 21, wherein the inflammatory disease is asthma, allergic rhinitis, atopic dermatitis, atheroma, atherosclerosis, or rheumatoid arthritis.

23. The method of claim 20, wherein the disease state is caused or associated with HIV infection.
